SEQUENCE LISTING (25 sequences)

SEQ ID NO: 1 - protein, 523 aa, Homo sapiens
MVAPGLVLGLVLPLILWADRSAGIGFRFASYINNDMVLQKEPAGAVIWGFGTPGATVTVTLRQGQETIMKKVTSVKAHSDTWMVVLDPMKPGGPFEVMAQQTLEKINFTLRVHDVLFGDVWLCSGQSNMQMTVLQIFNATRELSNTAAYQSVRILSVSPIQAEQELEDLVAVDLQWSKPTSENLGHGYFKYMSAVCWLFGRHLYDTLQYPIGLIASSWGGTPIEAWSSGRSLKACGVPKQGSIPYDSVTGPSKHSVLWNAMIHPLCNMTLKGVVWYQGESNINYNTDLYNCTFPALIEDWRETFHRGSQGQTERFFPFGLVQLSSDLSKKSSDDGFPQIRWHQTADFGYVPNPKMPNTFMAVAMDLCDRDSPFGSIHPRDKQTVAYRLHLGARALAYGEKNLTFEGPLPEKIELLAHKGLLNLTYYQQIQVQKKDNKIFEISCCSDHRCKWLPASMNTVSTQSLTLAIDSCHGTVVALRYAWTTWPCEYKQCPLYHPSSALPAPPFIAFITDQGPGHQSNVAK

SEQ ID NO: 2 - DNA, 2868 bp, Homo sapiens
cgggaggctgcgaggactgcaaaagggtggagtcggcctcgcccccgcccaggccccgcccctgccgggaacccactttcccagtcctaggcggcggtcagatccttgcaagcatggtcgcgccggggcttgtactcgggctggtgctgccattaatcctgtgggccgacagaagtgcaggtattggttttcgctttgcttcatacatcaataatgatatggtgctgcagaaggagcctgctggggcagtgatatggggcttcggtacacctggagccacagtgaccgtgaccctgcgccaaggtcaggaaaccatcatgaagaaagtgaccagtgtgaaagctcactctgatacgtggatggtggtactggatcctatgaagcctggaggacctttcgaagtgatggcacaacagactttggagaaaataaacttcaccctgagagttcatgacgtcctgtttggagatgtctggctctgtagtgggcagagtaacatgcagatgactgtgttacagatatttaatgctacaagggagttgtctaacactgcggcatatcagtctgtccgcatcctctctgtctctcccattcaagcagagcaggagctggaggaccttgttgcggttgacttgcagtggtctaagcccacctcagaaaacttaggccatggatatttcaagtacatgtcagcagtgtgctggctctttggacgtcacctttatgacactctgcagtatcccatcgggctgatcgcctccagctggggcgggacacccattgaagcctggtcatctggacggtcactgaaagcctgtggggtccctaaacaagggtccattccatacgattctgtaactggtcccagtaagcactctgttctctggaatgccatgatccatccactgtgcaatatgactctgaaaggggtagtatggtaccagggggagtccaatataaattataacacggatctgtacaattgcacattccctgcactcatcgaagactggcgtgaaaccttccaccgtggttcccaggggcagacggagcgtttcttcccatttggacttgtccagttatcttcagatttgtctaagaagagctcagacgatggatttccccagatccgttggcatcaaacagcagacttcggctatgtccccaacccaaagatgcccaatactttcatggctgtagctatggatctctgtgatagagactcgccttttggcagcatccaccctcgagataaacagactgtggcttatcggctgcatttgggggcccgtgctctggcttatggtgagaagaatttgacctttgaaggaccactgcctgagaagatagaactcttggctcacaaggggctgctcaatctcacatattaccagcaaatccaggtgcagaaaaaggacaacaagatatttgagatctcctgttgcagtgaccatcgatgcaagtggcttccagcttctatgaacaccgtctccacccagtccctgaccctggcgatcgattcttgtcatggcactgtggttgctctccgctatgcttggaccacgtggccttgtgaatataagcagtgtcccctataccaccccagtagtgccctgccagcccctcccttcattgctttcattacagaccagggtcctggacatcagagcaatgttgctaaatgactgtttcagtatgatcagaacttagatataaggatgggtccttcagattttagcatttaggagtttcaataataaccattgcttttaaaggaaattaatagaaagcctcattgaatggctttcagctagcacatggctgtttctatattctgatgagcccaggcttataggtaacttgaaatgcttgctttttgttccctagttggtctaagggtctgtattggactaattctgaactacagacaaattggacctcaatgtcatttacttccctcatattaatgggagtgaaatgtctaatacttttgcccctttttatccagagttgtgggaatctcaggattggaagagattttaaaggccacataggccagctagtgttcatgtgttctttataaaatttctcccatccaagtactaaccaggcccgaccctgcttagcttccgagatcagatgagatcaggcgcgttcagggtgatatggccgtagacgtctttacaaaattcctgacaggtggttactgaatctctctatgaactttccattcaaaactttccaagtttttccttatgtggaaccgaaatctttctttctcccgtgaaactttactactatcagataattgaagacagatctctttgtattctcttcaagcccaaaccaattctgttccttcaatctaaatagtggtaatatgaatgtttaagaaatgaaataagaaacatgtgcaggcactttggaaggtgctaagtgactgccctaaggaatgaaaagcaagggccaggtgggagtagcccagcgaaggcacttgggctgccaggaacaggaggcgtgggaaactctggcttaggaaaacatgaacacaggggcaacagaggcaaactgttgttcgagttaaatataaatctcaggctctttaaaggtaaaaggtttaaggataatccatttggaagaagaaaagagtgaggctgaaagtaaagccacatgacaagcatataaaaaaaaatgcagatgatacaaatatgaaagaggccttcagtgtttgtttattaagaatcttaatgcagtttactgatggattaaaaacagctaacattgtctgaaaattatgttacctataagaagttggaaataaataaaagcataatcactaaaaaaaaaaaaaaaaaaaaaaaaaaaaaa

SEQ ID NO: 3 - DNA, 25 bp, Artificial Sequence
  Oligonucleotide
tttaggagcaaggggttggccaaga

SEQ ID NO: 4 - DNA, 24 bp, Artificial Sequence
  Oligonucleotide
ggtttcctgaccttggcacaaggt

SEQ ID NO: 5 - DNA, 23 bp, Artificial Sequence
  Oligonucleotide
cggtacccggggatcaattcgag

SEQ ID NO: 6 - DNA, 22 bp, Artificial Sequence
  Oligonucleotide
gtacagcccagtcctgaggtcg

SEQ ID NO: 7 - DNA, 24 bp, Artificial Sequence
  Oligonucleotide
gacgcgagcccctagcctagctag

SEQ ID NO: 8 - DNA, 25 bp, Artificial Sequence
  Oligonucleotide
gactgtatgttctttgctgtttcac

SEQ ID NO: 9 - DNA, 25 bp, Artificial Sequence
  Oligonucleotide
tggataatgtgagctacagcattag

SEQ ID NO: 10 - DNA, 25 bp, Artificial Sequence
  Oligonucleotide
cagaggctgggaatagccacaaatg

SEQ ID NO: 11 - DNA, 25 bp, Artificial Sequence
  Oligonucleotide
agaaacagccctgcttctccttgtt

SEQ ID NO: 12 - DNA, 25 bp, Artificial Sequence
  Oligonucleotide
gtggatcaaggtgattcagtgcagc

SEQ ID NO: 13 - DNA, 25 bp, Artificial Sequence
  Oligonucleotide
cctgaaatagtcaccatcaggaagg

SEQ ID NO: 14 - DNA, 25 bp, Artificial Sequence
  Oligonucleotide
gacctctgcccacccttctcacctc

SEQ ID NO: 15 - DNA, 25 bp, Artificial Sequence
  Oligonucleotide
tgggacattcaccatatacttaact

SEQ ID NO: 16 - DNA, 25 bp, Artificial Sequence
  Oligonucleotide
acaagtgaaagtggatagatgacca

SEQ ID NO: 17 - DNA, 25 bp, Artificial Sequence
  Oligonucleotide
tgttgctttaagccactacgttcta

SEQ ID NO: 18 - DNA, 25 bp, Artificial Sequence
  Oligonucleotide
aagaccactacctcagggcatggat

SEQ ID NO: 19 - DNA, 25 bp, Artificial Sequence
  Oligonucleotide
caggaaagagatccaaatagcacat

SEQ ID NO: 20 - DNA, 25 bp, Artificial Sequence
  Oligonucleotide
gcaatgagagaagcagaagcagagt

SEQ ID NO: 21 - DNA, 25 bp, Artificial Sequence
  Oligonucleotide
gactcttaagtgccaatcctcagtc

SEQ ID NO: 22 - DNA, 25 bp, Artificial Sequence
  Oligonucleotide
ctgatcacagagttcagtcaacttt

SEQ ID NO: 23 - DNA, 25 bp, Artificial Sequence
  Oligonucleotide
caaccaagacccgccacatcgtaat

SEQ ID NO: 24 - DNA, 25 bp, Artificial Sequence
  Oligonucleotide
caggtttgttctgcttactgtagga

SEQ ID NO: 25 - DNA, 25 bp, Artificial Sequence
  Oligonucleotide
atatagaaacagccatgtgctagct

(19) United States
(12) Patent Application Publication
(10) Pub. No.: US 2011/0311507 A1
(43) Pub. Date: Dec. 22, 2011

(54) Methods for Diagnosing and Treating Autoimmune Disorders

(75) Inventors: Shiv Pillai, Lexington, MA (US); Kendra N. Taylor, Chelsea, MA (US); Cariappa Annaiah, Milford, MA (US)

(73) Assignee: THE GENERAL HOSPITAL CORPORATION, Boston, MA (US)

(21) Appl. No.: 13/140,295

(22) PCT Filed: Dec. 15, 2009
(86) PCT No.: PCT/US09/68020

§ 371 Date: Sep. 7, 2011

Related U.S. Application Data
(60) Provisional application No. 61/139,945, filed on Dec. 22, 2008.

(51) Int. Cl. A61K 38/46; G01N 33/53; G01N 21/64; C12Q 1/68; A61P 29/00; A61P 37/00; A61P 3/10; A61P 5/14; A61P 19/04; A61P 1/00; A61P 19/02; C12Q 1/02; A61P 25/00

(52) U.S. Cl. 424/94.6; 435/29; 435/7.92; 435/7.21; 435/6.12

(57) ABSTRACT

Provided herein are, inter alia, methods of identifying patients who are at risk of developing an autoimmune disorder, and treating patients suffering from autoimmune disorders.

### RELATED APPLICATIONS

This application claims the benefit of U.S. Provisional Patent Application Ser. No. 61/139,945, filed on Dec. 22, 2008, the entire contents of which are hereby incorporated by reference.

### STATEMENT OF GOVERNMENT SUPPORT

This work was supported in part by NIH Grant Numbers AI064930, CA102793 and AI670505. The government has certain rights to the invention.

### TECHNICAL FIELD

This invention relates to methods of identifying patients who are at risk of developing an autoimmune disorder, and treating patients suffering from autoimmune disorders.

### BACKGROUND

Autoimmune disorders result from an aberrant immune response. The proper diagnosis of many autoimmune diseases is nontrivial, often relying on examination of family health history and/or physical examination. Some autoimmune diseases may be detected through more sophisticated methods, e.g. autoantibody and immunofluorescent assays, but such methods are not effective for diagnosing the majority of these diseases. Furthermore, treatments for autoimmune diseases can often impair a subject's lifestyle through dietary manipulation and restriction as well as increasing one's susceptibility to infection, a common occurrence in immunosuppressive therapeutic strategies.

### SUMMARY

In one aspect, disclosed herein are uses of an SIAE polypeptide in the manufacture of a medicament for the treatment of an autoimmune disorder in a subject.

In another aspect, described herein are uses of an SIAE polypeptide for the treatment of an autoimmune disorder in a subject.

In another aspect, provided herein are methods for treating an autoimmune disorder in a subject, the method comprising (a) identifying a subject in need of such treatment; and (b) administering to the subject a therapeutically effective amount of an SIAE polypeptide.

The SIAE polypeptide useful for the methods and uses described herein can comprise the amino acid sequence of SEQ ID NO:1. The subject can have a defect in an SIAE function (e.g., a defect in catalytic activity of SIAE or secretion of SIAE). The subject can also have an SIAE gene that encodes a functionally defective SIAE polypeptide.

The autoimmune disorder can include rheumatoid arthritis, type I diabetes, systemic lupus erythematosus, inflammatory bowel disease, multiple sclerosis, juvenile idiopathic arthritis, autoimmune thyroiditis, and sjogren's syndrome.

The methods for treating an autoimmune disorder can further comprise determining whether the subject has a defect in an SIAE function by determining the presence of an SIAE variant allele that encodes a functionally defective SIAE protein, wherein the presence of the variant allele indicates that the subject has a defect in an SIAE function.

The methods can also further comprise determining whether the subject has a defect in an SIAE function by obtaining a level of 9-O-acetyl sialic acid on B cells in the subject, wherein an increase in the level in the subject as compared to a level in a control subject (or to a pre-determined control level) indicates that the subject has a defect in an SIAE function.

Additionally, the methods can further comprise determining whether the subject has a defect in an SIAE function by obtaining a serum level of SIAE protein in the subject, wherein a decrease in the level in the subject as compared to a level in a control subject (or to a pre-determined control level) indicates that the subject has a defect in an SIAE function.

In yet another aspect, provided herein are methods for determining whether a subject is at risk for developing an autoimmune disorder. The methods comprise (a) sequencing all or a part of a sialic acid acetylesterase (SIAE) gene of a subject; (b) determining a subject amino acid sequence encoded by the SIAE gene in the subject; (c) comparing the subject amino acid sequence to a reference SIAE amino acid sequence; and (d) if there is an amino acid change in the subject amino acid sequence as compared to the reference SIAE amino acid sequence, determining an activity of an SIAE polypeptide comprising the subject amino acid sequence; wherein a decrease in the activity indicates that the subject is at risk for developing an autoimmune disorder.

In one aspect, described herein are methods for determining whether a subject is at risk for developing an autoimmune disorder. The methods comprise (a) providing a test sample from a subject; and (b) assaying a level of 9-O-acetyl sialic acid on the surface of B cells in the test sample; wherein an increase in the level in the test sample as compared to a level in a control sample (or to a pre-determined control level) indicates that the subject is at risk for developing an autoimmune disorder.

In another aspect, provided herein are methods for determining whether a subject is at risk for developing an autoimmune disorder, the methods comprising (a) providing a test serum or plasma sample from a subject; and (b) assaying a level of SIAE protein in the sample; wherein a decrease in the level in the test sample as compared to a level in a control sample (or to a pre-determined control level) indicates that the subject is at risk for developing an autoimmune disorder.

The term “suitable for treatment with SIAE compositions” as used herein, when used in reference to a subject refers to subjects who are more likely to benefit from treatment with compositions comprising SIAE than a subject selected randomly from the population.

The term “enzyme,” as used herein, refers to proteins that catalyze, i.e. accelerate, chemical reactions. In enzymatic reactions, the molecules at the beginning of the process are called substrates, and the enzyme converts them into different molecules, the products. Enzymes are extremely selective for their substrates and speed up only a few reactions from among many possibilities, the set of enzymes made in a cell determines which metabolic pathways occur in that cell. One example of an enzyme is an esterase, a hydrolase enzyme that splits esters into an acid and an alcohol in a chemical reaction with water called hydrolysis. A wide range of different esterases exists that differ in their substrate specificity, their protein structure, and their biological function. The present invention contemplates a specific esterase, sialate:O-acetyl esterase, which an enzyme that catalyzes the following chemical reaction:

N-acetyl-O-acetylneuraminate+H₂O [symbol] N-acetylneuraminate+acetate

The two substrates of this enzyme are N-acetyl-O-acetylneuraminate and H₂O and its two products are N-acetylneuraminate and acetate. The enzyme belongs to the family of hydrolases, specifically those acting on carboxylic ester bonds. The systematic name of this enzyme class is N-acyl-O-acetylneuraminate O-acetylhydrolase. Other names in common use include N-acetylneuraminate acetyltransferase, sialate 9(4)-O-acetylesterase, and sialidase.

The term “at risk for” as used herein, refers to a medical condition or set of medical conditions exhibited by a patient which may predispose the patient to a particular disease or affliction. For example, these conditions may result from influences that include, but are not limited to, genetic, behavioral, emotional, chemical, biochemical, or environmental influences.

The term “pharmaceutically” or “pharmacologically acceptable,” as used herein, refer to molecular entities and compositions that do not produce adverse, allergic, or other untoward reactions when administered to an animal or a human.

The term, “pharmaceutically acceptable carrier,” as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). A biological sample suspected of containing nucleic acid may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1(a) is a set of two graphs showing that an in-frame deletion of exon 2 in a murine Lse complementary DNA resulted in a protein that lacked esterase activity. A wild-type C-terminal FLAG-tagged murine SIAE expression construct of the Lse form and an exon 2 deleted version of this cDNA were transfected in COS 7 cells. Cell culture medium was recovered and the recombinant protein was purified by an antibody column directed to the FLAG epitope. Western blotting analysis on eluate fractions showed that both proteins were expressed, bound and eluted from the column. Column eluate fractions were subjected to an assay for sialic acid 9-O-acetylesterase activity (i) and acetyl-CoA esterase activity (ii).

FIG. 1(b) includes (i) a representation of the targeting construct used to specifically target exon 2 of Lse, (ii) the expected structure of the locus after the targeting; (iii) a reproduction of a Southern blot on ES cells at each step of manipulation detected by a loxP specific probe; and (iv) a reproduction of a Southern blot of mouse-tail DNA after mating heterozygous mice that also expressed the ZP3-Cre transgene. The expected deleted fraction is 6.5 Kb and the expected WT fragment is 4.2 Kb.

FIG. 1(c) is a set of scatter plots and line graphs showing that BCR signaling was accelerated and enhanced in Siae mutant mice. Splenocytes from mutant and wild type mice were loaded with Indo-1, stained with follicular and marginal zone (MZ) B cell phenotype markers and stimulated with F(ab′)₂ anti-IgM in the presence of 2.4G2. Populations gated on include newly formed (NF/T1), follicular (FO), and marginal zone B cells and their precursors (MZ and MZP). Results are representative of three separate experiments.

FIG. 2(a) is a reproduction of an immunohistochemistry staining showing a decrease in MZ B cells in SIAE^{Δ2/Δ2} spleens. B cells were stained with anti-IgM and metallophilic macrophages with the MOMA-1 antibody. MZ—marginal zone. FO—follicular compartment. MZMM—marginal zone metallophilic macrophages.

FIG. 2(b) is a set of plots showing that flow cytometric analyses revealed a reduction in MZ B cells in SIAE^{Δ2/Δ2} mice. Follicular B cells were also slightly decreased but to a lesser degree. Results are representative of more than 10 mice per group.

FIG. 2(c) is a set of plots showing that perisinusoidal bone marrow B cells were reduced in SIAE^{Δ2/Δ2} mice as revealed by flow cytometry. Results are representative of 5 mice per group.

FIG. 2(d) is a set of plots showing that peritoneal B-1b B cell numbers were increased in SIAE^{Δ2/Δ2} mice. Flow cytometric analyses were performed on peritoneal cavity B cells. Results are representative of 5 mice per group.

FIG. 2(e) is a set of plots showing that SIAE affected B cell development in a B cell intrinsic manner. Rag-1^{-/-} mice were reconstituted with mutant or wild type bone marrow and spleen and bone marrow B cells were analyzed 7 weeks later in the reconstituted mice. Results are representative of 3 mice per group.

FIG. 2(f) is a set of plots showing that T cell development in the thymus and peripheral T cells in the spleen were unaffected in SIAE^{Δ2/Δ2} mice. Thymocyte and splenic populations were analyzed by flow cytometry in wild type and SIAE^{Δ2/Δ2} mice. Results are representative of 3 mice per group.

FIG. 3(a) is a reproduction of an immunoblot showing that recruitment of SHP-1 by CD22 in SIAE mutant mice was decreased. Splenocytes from mutant and wild-type mice were stimulated via the BCR, immunoprecipitated with antibody to CD22, and Western blots were developed with antibodies to SHP-1, CD22 and phosphotyrosine.

FIG. 3(b) is a set of plots showing that hyperacetylation at the 9-O-position of sialic acid was seen in Siae mutant B cells. Splenocytes were reacted with the CHE-FcD reagent and then stained with surface phenotype markers. CHE-FcD staining is normally seen on CD4 T cells (represented here as IgM⁻CD21⁻ cells, extreme left panel).

FIG. 3(c) is a dot plot showing that serum immunoglobulins of specific isotypes were increased in SIAE^{Δ2/Δ2} mice. Immunoglobulin isotypes were quantitated by ELISA. Each data point represents a mouse. Horizontal bars represent the mean.

FIG. 3(d) is a dot plot showing that levels of autoantibodies in the serum of SIAE^{Δ2/Δ2} mice were increased. ELISA was used to estimate the levels of various autoimmune antibodies in serum. Each data point represents a mouse. Horizontal bars represent the mean.

FIG. 3(e) shows that Siae mutant mice exhibited histological features of glomerulonephritis. Paraffin embedded sections of kidney from mutant and wild type mice were stained with hematoxylin and eosin (upper panel) and with periodic acid Schiff (PAS) reagent (lower panel). The glomeruli of the mutant mice (upper right panel) show mesangial hypercellularity, and expansion due to PAS positive deposits (lower panel).

FIG. 3(f) is a reproduction of a set of fluororescence images showing IgG immune complex deposition in glomeruli of SIAE^{Δ2/Δ2} mice. Immunofluorescence studies on frozen sections of kidney reveal enlarged glomeruli with IgG positive deposits in the mesangium in SIAE^{Δ2/Δ2} mice. The magnification is marked on each image. Bars −100 μm. These sections are from the same mice as those shown in FIG. 3(e).

FIG. 4(a) is a set of plots showing non-conservative substitutions in SIAE in patients with autoimmune disease. The SIAE gene was sequenced in 19 patients with high serum anti-nuclear antibody (ANA) titers and 190 ethnically matched healthy controls. 12 of the 19 patients had a diagnosis of autoimmunity and two of these 12 patients had non-conservative changes in SIAE (T312M and K400N). These changes were not seen in 190 controls.

FIGS. 4(b) and 4(c) are a set of immunoblots and bar graphs showing that T312M SIAE accumulated in cells but was poorly secreted. A murine Siae FLAG-tagged cDNA in the pCDNA 3.1 expression vector was engineered to generate a catalytic site mutation (S127A) as well as the two substitutions (T312M/T337M and K400N/K425N) seen specifically in autoimmune disease patients. 293 T cells were transfected with individual cDNAs and cell lysates were divided into two portions, one immunoprecipitated with anti-Flag antibodies for a catalytic assay (see FIG. 4(c)) and the other immunoprecipitated similarly but then separated by SDS PAGE, transferred and analyzed for expression by an anti-FLAG Western blot assay (see FIG. 4(b)). In order to assess secretion, culture supernatants of transfected cells were also immunoprecipitated and examined by a Western blot approach for the expression of wild type and mutant SIAE.

FIG. 4(d) is a representation of some proteins involved in BCR signaling.

FIG. 5 is a graph demonstrating that SIAE mutant splenocytes proliferated more than wild type B cells.

FIG. 6 is a set of graphs showing that surface expression of CD22 on splenocytes in SIAE^{Δ2/Δ2} mice was unaltered compared to wild type mice. The results shown are representative of three mice per group.

FIG. 7 is a set of graphs showing antibody responses to synthetic T-dependent and T-independent antigens in SIAE mutant and wild type mice. Five mice were analyzed in each group. Error bars represent standard error of the mean.

FIG. 8 is an examplary human SIAE protein sequence (Accession No NP 733746; SEQ ID NO: 1)

FIG. 9 is an examplary human SIAE nucleotide sequence encoding SEQ ID NO:1 (Accession No. NM_{—}170601; SEQ ID NO: 2).

FIG. 10 is a set of graphs demonstrating SIAE modulation of calcium influx in three mouse splenocyte cell lines. Top Panel: NFT1 (IgM^{hi}CD23^{lo-neg}); Middle Panel: FO-II (IgM^{hi}CD21^{int}); Bottom Panel: FO-II, IgM^{lo}CD21^{int}. WT+Flag: Normal calcium flux in activated wild type B cells in the presence of Flag protein (5 μg). SIAE^{Δ2/Δ2}+Flag: Enhanced calcium flux in activated Siae knockout B cells (SIAE^{Δ2/Δ2}) in the presence of Flag protein (5 μg). SIAE^{Δ2/Δ2}+Flag+SIAE: Rescued calcium influx in activated Siae knockout B cells (SIAE^{Δ2/Δ2}) in the presence of Flag protein (4 μg), and SIAE protein (6 μg).

FIG. 11 is a set of bar graphs demonstrating the changes in SHP-1/CD22 ratios of data collected in accordance with the gel densitometry experiments presented in FIG. 3.

FIG. 12 is a set of graphs demonstrating lymphoid gating responses in 11 week old splenocyte cells treated with the CHE-FcD reagent and stained with surface phenotype markers. Results are representative of three mice. X-axis: log of fluorescence intensity.

FIG. 13 is a set of bar graphs and a representation of a set of immunoblots showing the activities of various SIAE variants. Each SIAE variant found in subjects with autoimmunity was re-created by site-directed mutagenesis in a human SIAE cDNA, which was then sequenced along its entire length. Wild type (WT) SIAE, a known catalytic site mutant (S127A SIAE), and each SIAE variant that was unique to autoimmune subjects were transfected into 293T cells. Assays were performed for A3G SIAE, N33S SIAE, C196F SIAE, G2121R SIAE, C266G SIAE, Q309P SIAE, T312M SIAE, Y349C SIAE, K400N SIAE, F404S SIAE, and R479CSIAE. Quantitative western blot analysis (using anti-FLAG antibodies) was performed on both the cell lysate and the culture supernatant, and a ratio of these two measurements is shown in the right hand panels of the figure. “Mock” refers to cells which were not transfected but from which lysate and supernatant were analyzed. Half of each lysate was immunoprecipitated and examined for esterase activity, presented following normalization for lysate SIAE protein content. Each row shows results from one representative transfection. Each variant was tested in this manner on at least three or more occasions to ensure reproducibility.

FIG. 14 is a set of bar graphs and a reproduction of immunoblots showing the activities of various SIAE variants. Each variant identified in control subjects was recreated in an SIAE cDNA as described above for subjects with autoimmunity. Wild type (WT) SIAE, S127A SIAE and each SIAE variant that was unique to controls (R62H SIAE, G64S SIAE, Q161K SIAE, R314H SIAE, H447R SIAE, M456I SIAE, and Q462R SIAE) was transfected into 293T cells. Also shown are results from M89V SIAE, which was found in heterozygous form in both patients and controls and in homozygous form only in patients. T312M SIAE was observed in one control and in two patients. Results for this variant are included in FIG. 13. Analyses were performed as described in the legend for FIG. 13.

FIG. 15(a) is set of a bar graph and a reproduction of immunoblots showing that Murine C196F SIAE, and the murine equivalents of Q309P SIAE and T 312M SIAE, (Q335P and T338M SIAE), function in a dominant interfering fashion but M89V SIAE does not. V5 tagged wild type SIAE was transfected along with FLAG-tagged C196F SIAE or FLAG tagged M89V SIAE and the enzyme activity of V5 tagged wild type SIAE was assessed in transfectants as a function of its protein level. Expression of mutant SIAE was monitored by an anti-FLAG Western blot of immunoprecipitated mutant proteins.

FIG. 15(b) is a reproduction of a set of immunoblots showing pulse-chase analysis comparing secretion of wild type SIAE and M89V SIAE. Transfected 293T cells were metabolically pulse-labeled with ³⁵[S]methionine and lysates and supernatants were immunoprecipitated with anti-FLAG antibodies after 10 minutes, 1 hour, 2 hours and 4 hours of chase. Proteins were separated by SDS-PAGE and revealed by autofluorography. The position of molecular weight markers is indicated on the left in kilodaltons.

FIG. 16(a) is a set of a bar graph and a reproduction of immunoblots showing that murine G212R Siae, and the murine equivalents of Y349C, F404S and R479CSIAE, (Y375C, F430S and R505CSiae), function in a dominant interfering fashion but M89V Siae does not. V5-tagged wild type Siae was transfected along with FLAG-tagged mutant Siae molecules and the enzyme activity of V5-tagged wild type Siae was assessed in transfectants as a function of its protein level. Expression of mutant Siae was monitored by an anti-FLAG Western blot of immunoprecipitated mutant proteins.

FIG. 16(b) is a set of a bar graph and a reproduction of an immunoblot showing that the murine equivalent of K400N (K426N) lacks dominant negative activity. V5-tagged WT Siae was transfected along with FLAG-tagged WT Siae or FLAG-tagged K426N Siae and the enzyme activity of V5-tagged WT Siae was assessed in transfectants as a function of its protein level, determined using a quantitative immunoblot assay.

FIGS. 17(a) and (b) is a reproduction of a set of immunoblots showing that wildtype Siae associates with itself and with functionally defective Siae variant proteins. (a) FLAG-tagged wild type murine Siae and wild type V5 tagged Siae were transfected together into 293T cells, and reciprocal immunoprecipitation/Western blot assays were performed on cell lysates with anti-V5 antibodies and anti-FLAG antibodies. (b) Mutant FLAG-tagged murine Siae and a V5-tagged wild type Siae were co-transfected into 293T cells and immunoprecipitation/western blot assays were performed. DT refers to double transfection. S and L refer to supernatant and lysate respectively.

FIGS. 18(a) and (b) are plots showing that patients with SIAE mutations and controls share a common ancestry. (a) A principal components analysis was run with markers informative for major ancestry, and show three separate groups (crosses) for HAP map samples: The Caucasian cluster localizes with almost all controls (black circles) and SIAE cases (grey circles). The African cluster is at upper right, Asian cluster below. (b) A second analysis using only Caucasian clustered samples shows that the cases and controls cluster similarly using markers that capture European diversity; most of this diversity relates to north-south ancestry differences, giving the spread along the x-axis in the plot.

FIG. 19 is a reproduction of an immunoblot showing that SIAE is secreted from stably transfected U2OS cells.

FIG. 20 is a plot showing 9-O-acetylation of sialic acid on the surface of CD19+ human B cells from peripheral blood.

FIG. 21 is a plot showing that some SLE patients tested exhibited increased 9-O-acetylation of sialic acid on B cells as compared to normal subjects. The level of 9-O-acetylated sialic acid on B cells was measured by the CHE-Fc-D technique used in FIG. 20. The degree of a shift to the right when using CHE-Fc-D and the labeled second antibody as compared with the second antibody alone was quantitated for both controls and Lupus subjects and plotted as mean fluorescence intensity.

FIG. 22 is a reproduction of an immunoblot showing that SIAE was detected in human serum.

### DETAILED DESCRIPTION

A large number of autoimmune disorders have a major B cell component. Many of these disorders, but not all, respond to B cell depletion therapy. The challenge is to be able to rationally identify subsets of patients who harbor an underlying defect in a specific pathway of B cell tolerance, and who are candidates to be rationally treated with novel directed therapies. A number of susceptibility loci for human autoimmune disorders have been uncovered by genome wide association studies, and the Odds Ratios for these associations are generally modest (Cohen et al., Science 305: 869-72 (2004); Gregersen & Behrens, Nat Rev Genet. 7: 917-28 (2006)). None of the loci uncovered by these studies so far involves a human gene that can be functionally linked to a loss of B lymphocyte tolerance in any common autoimmune disorder.

Sialic acid acetylesterase (SIAE) was identified previously as a gene that is up-regulated during B cell maturation (Stoddart et al., Nucleic Acids Res. 24:4003-4008 (1996)). Evidence provided herein demonstrate that SIAE is an enzymatic regulator of B cell tolerance. Further, data described herein reveal that loss-of function mutations in SIAE contribute to autoimmune disease susceptibility in a significant manner.

Accordingly, the present invention provides, inter alia, novel methods for identifying subjects who are suffering from an autoimmune disorder or is at risk of developing an autoimmune disorder. The present invention also provides methods for treating autoimmune disorders.

I. Autoimmune Diseases

As used herein, an autoimmune disorder is a condition that occurs when the immune system mistakenly attacks and destroys healthy body tissue. An autoimmune disorder may result in tissue changes including, but not limited to, the destruction of one or more types of body tissue, abnormal growth of an organ, or changes in organ function. Further, an autoimmune disorder may affect one or more organ or tissue types. Organs and tissues commonly affected by autoimmune disorders include, but are not limited to, red blood cells, blood vessels, connective tissues, endocrine glands such as the thyroid or pancreas, muscles, joints, or skin.

Examples of autoimmune (or autoimmune-related) disorders include, but are not limited to Hashimoto's thyroiditis, pernicious anemia, Addison's disease, type I diabetes mellitus type I, rheumatoid arthritis, systemic lupus erythematosus, dermatomyositis, Sjogren syndrome, lupus erythematosus, multiple sclerosis, myasthenia gravis, reactive arthritis, Grave's disease, celiac disease, Crohn's disease, acute disseminated encephalomyelitis, ankylosing spondylitis, antiphospholipid antibody syndrome, aplastic anemia, autoimmune hepatitis, autoimmune oophoritis, celiac disease, gestational pemphigoid, Goodpasture's syndrome, Guillain-Barré syndrome, idiopathic thrombocytopenic purpura, Kawasaki's disease, opsoclonus myoclonus syndrome, optic neuritis, Ord's thyroiditis, pemphigus, polyarthritis, primary biliary cirrhosis, PSORIASIS, Reiter's syndrome, small vessel vasculitis, Takayasu's arteritis, temporal arteritis, ulcerative colitis, warm autoimmune hemolytic anemia, or Wegener's granulomatosis.

Generalized tests are sometimes performed to diagnose an autoimmune disorder, including, but not limited to, erythrocyte sedimentation rate (ESR), or C-reactive protein (CRP).

Current treatment of autoimmune diseases are usually directed to reducing symptoms and controlling the autoimmune process while maintaining the body's ability to fight disease. Treatments vary widely and depend on the specific disease and specific symptomology. For example, some patients may need supplements to replenish a hormone or vitamin that the body is lacking, such as thyroid supplements, vitamins, or insulin injections. However, if the autoimmune disorder affects the blood, an affected patient may need blood transfusions. Other medications may be used such as those to help with movement or other functions for autoimmune disorders that affect the bones, joints, or muscles. Medicines that control or reduce the immune system's responsivity (i.e., for example, immunosuppressants) are also prescribed. Such immunosuppressive medicines may include, but are not limited to, corticosteroids and immunosuppressant drugs such as cyclophosphamide or azathioprine. Autoimmune prognosis is dependent on the specific disease. Many autoimmune diseases are chronic and can be controlled with treatment.

II. Sialic Acids and Sialic Acid Acetylesterase

Sialic Acids

The term “sialic acid” refers to a compound of the following general chemical formula:

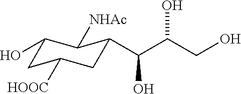

Sialic acid is a generic term for the N- or O-substituted derivatives of neuraminic acid, a nine-carbon monosaccharide. It is not intended that the invention be limited by the number or classification of substituents that may be connected to a sialic acid moiety. The most common member of this group, N-acetylneuraminic acid is also referred to as Neu5Ac or NANA. Sialic acids are found widely distributed in animal tissues and in bacteria, especially in glycoproteins and gangliosides. The amino group often bears either an acetyl or a glycolyl group. It is not intended that the type of chemical substituent found in this position limit the present invention. The hydroxyl substituents are typically selected from acetyl, lactyl, methyl, sulfate, and phosphate groups. It is not intended that the type of chemical substituent found in this position limit the present invention.

Sialic acids are believed to be nine-carbon monosaccharides involved in intercellular communication and recognition of toxins and viruses. Sialic acids also may mediate a multitude of functions in intercellular communication as well as pathogen detection and response. Acetylation of sialic acid on the 9-OH position represents an abundant postsynthetic carbohydrate modification in vertebrates. The hemagglutinins of influenza C viruses and certain nidoviruses including group 2 coronaviruses recognize 9-O-acetylated sialic acid containing glycoconjugates on the surface of host cells, but the biological function in the host of this carbohydrate modification has remained unknown.

The structural complexity of sialic acids may have evolved in part to enhance the range of recognition events that involve glycans during mammalian development, cell migration, and host defense. Sialic acids may differ structurally from other monosaccharides by the presence of an acidic carboxyl group, an N-acyl substituent, and an exocyclic glycerol side chain.

For example, one structural variation of sialic acid comprises a 5-N-acyl group (i.e., an N-acyl group in the 5-position). One abundant form of sialic acid is 5-N-acetylneuraminic acid (Neu5Ac). In normeural tissues of most mammalian species (but not in humans) a proportion of the biosynthetic intermediate, CMP-Neu5Ac, is converted by a specific hydroxylase into the N-glycolyl form (NeuSGc). While N-acylation is generally stable and not suited for kinetic modulation of signaling events, a post-synthetic modification that has the potential to dynamically influence cell signaling events is the O-acetylation of sialic acids. A common type of O-acetylation event of hitherto unknown function comprises modification of α2-6-linked sialic acid containing glycoconjugates at the 9-OH position, or at the 7-OH position from which the acetyl group can spontaneously, and rapidly, migrate to the 9-OH position.

The 7-OH/9-OH acetylation event modifies a carbohydrate structure that is recognized by certain Siglecs (i.e., for example, sialic acid binding Ig domain containing lectins). Siglecs comprise a large group of vertebrate sialic acid-recognizing cell surface proteins. The Siglec family includes, but is not limited to, thirteen members in humans including, sialoadhesin (Siglec-1), CD22 (Siglec-2), CD33 (Siglec-3), myelin associated glycoprotein (Siglec-4), and the so-called CD33-related Siglecs. Some reports have indicated that CD22 acts as a negative regulator of B cell antigen receptor signaling.

Sialic Acid Acetylesterase (Proteins, Nucleic Acids, Vectors and Host Cells)

Sialic acid acetylesterase or sialate:O-acetyl esterase (SIAE) is molecularly characterized as an enzyme that removes acetyl groups from the 9-OH position of α2-6-linked sialic acid (Guimaraes et al., J. Biol. Chem. 271: 13697-13705 (1996)). The SIAE gene may generate at least two splice variants: i) the so-called lysosomal sialyl acetyl esterase (Lse) comprising a signal peptide thereby allowing entry into a secretory pathway; and ii) the so-called cytosolic esterase (Cse) that lacks a signal peptide. Although the Lse protein was once thought to localize only within lysosomes, data disclosed herein show that it is secreted.

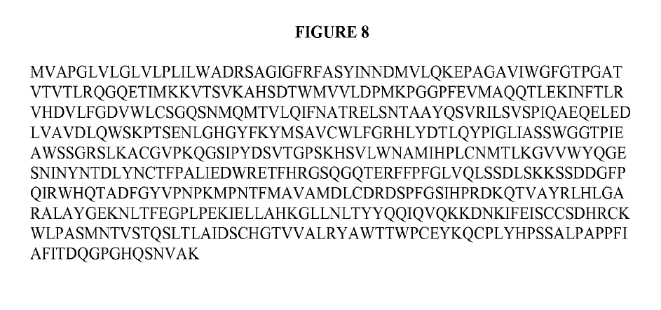

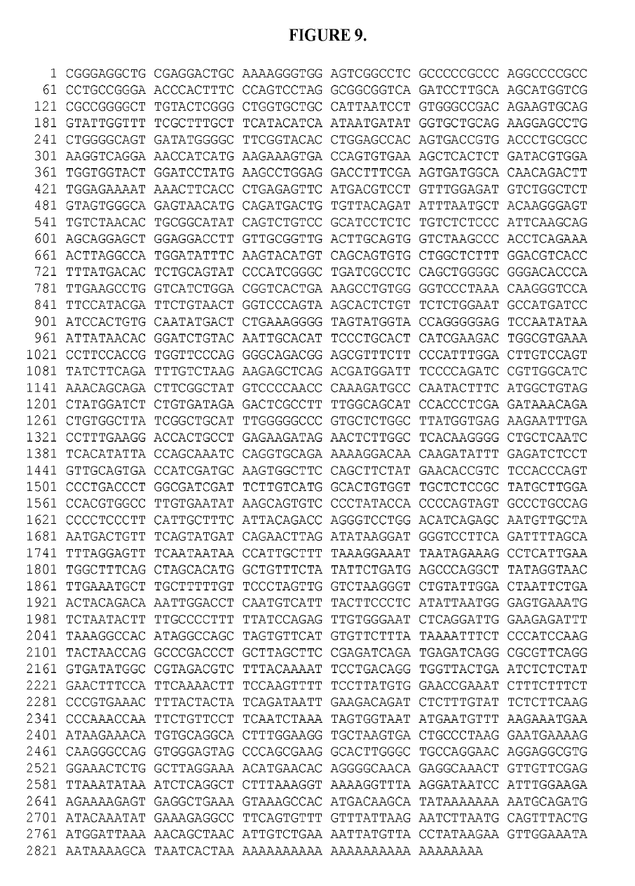

SIAE polypeptides or biologically active fragments thereof, and nucleic acids encoding full-length SIAE polypeptides or biologically active fragments thereof are useful for the diagnostic and treatment methods described herein. SIAE polypeptides and nucleic acids encoding them are readily obtained by one of ordinary skill in the art without undue experimentation. For example, SEQ ID NO:1 is an examplary amino acid sequence of a full-length human SIAE polypeptide (Accession No. NP_{—}733746; shown in FIG. 8). Full-length SIAE nucleic acids include human SIAE nucleic acid sequence, such as SEQ ID NO: 2 (Accession No. NM_{—}170601; shown in FIG. 9). SEQ ID NO:1 is also referred to herein as the reference or wildtype human SIAE amino acid sequence. A biologically active or functional fragment of SIAE can be a fragment that includes the catalytic domain of SIAE.

The terms “protein” and “polypeptide” both refer to any chain of amino acids, regardless of length or post-translational modification (e.g., glycosylation or phosphorylation). Thus, the terms “SIAE protein” and “SIAE polypeptide” include full-length naturally occurring isolated proteins, as well as recombinantly or synthetically produced polypeptides that correspond to the full-length naturally occurring proteins, or to a fragment of the full-length naturally occurring or synthetic polypeptide.

As discussed above, the term “SIAE polypeptide” includes both biologically active and non-biologically active fragments of naturally occurring or synthetic SIAE polypeptides. Fragments of a protein can be produced by any of a variety of methods known to those skilled in the art, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid that encodes the polypeptide. Expression of such mutagenized DNA can produce polypeptide fragments. Digestion with “end-nibbling” endonucleases can thus generate DNAs that encode an array of fragments. DNAs that encode fragments of a protein can also be generated, e.g., by random shearing, restriction digestion, chemical synthesis of oligonucleotides, amplification of DNA using the polymerase chain reaction, or a combination of the above-discussed methods. Fragments can also be chemically synthesized using techniques known in the art, e.g., conventional Merrifield solid phase FMOC or t-Boc chemistry. For example, peptides of the present invention can be arbitrarily divided into fragments of desired length with no overlap of the fragments, or divided into overlapping fragments of a desired length.

A purified or isolated compound is a composition that is at least 60% by weight the compound of interest, e.g., an SIAE polypeptide or antibody. Typically, the preparation is at least 75% (e.g., at least 90%, 95%, or 99%) by weight the compound of interest. Purity can be measured by any appropriate standard method, e.g., column chromatography, polyacrylamide gel electrophoresis, or HPLC analysis.

In certain embodiments, SIAE polypeptides include sequences substantially identical to all or portions of a naturally occurring SIAE polypeptides. Polypeptides “substantially identical” to the SIAE polypeptide sequences described herein have an amino acid sequence that is at least 65% (e.g., at least 75%, 80%, 85%, 90%, 95%, 99% or 99.9%, e.g., 100%), identical to the amino acid sequences of the SIAE polypeptides represented by SEQ ID NO:1 (measured as described herein). For purposes of comparison, the length of the reference SIAE polypeptide sequence is typically at least 20 amino acids, e.g., at least 30 or 50 amino acids.

In the case of polypeptide sequences that are less than 100% identical to a reference sequence, the non-identical positions are preferably, but not necessarily, conservative substitutions for the reference sequence. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

SIAE polypeptides useful in the methods and compositions described herein include, but are not limited to, recombinant polypeptides and natural polypeptides. Also included are nucleic acid sequences that encode forms of SIAE polypeptides in which naturally occurring amino acid sequences are altered or deleted. Certain nucleic acids of the present invention may encode polypeptides that are soluble under normal physiological conditions.

Also within the invention are nucleic acids encoding fusion proteins, and the fusion proteins themselves, in which an SIAE polypeptide is fused to an unrelated polypeptide, also referred to herein as a “heterologous polypeptide” or a “non-SIAE polypeptide” (e.g., a marker polypeptide or a fusion partner) to create a fusion protein. For example, the polypeptide can be fused to a hexa-histidine tag or a FLAG tag to facilitate purification of bacterially expressed polypeptides or to a hemagglutinin tag or a FLAG tag to facilitate purification of polypeptides expressed in eukaryotic cells. The invention also includes, for example, isolated polypeptides (and the nucleic acids that encode these polypeptides) that include a first portion and a second portion, where the first portion includes, e.g., an SIAE polypeptide, and the second portion includes an immunoglobulin constant (Fc) region or a detectable marker (e.g., β-galactosidase, invertase, green fluorescent protein, luciferase, chloramphenicol acetyltransferase, beta-glucuronidase, exo-glucanase, and/or glucoamylase).

The fusion partner can be, for example, a polypeptide that facilitates secretion, e.g., a secretory sequence. Such a fused polypeptide is typically referred to as a preprotein. The secretory sequence can be cleaved by the host cell to form the mature protein. Also within the invention are nucleic acids that encode an SIAE polypeptide fused to a polypeptide sequence to produce an inactive preprotein. Preproteins can be converted into the active form of the protein by removal of the inactivating sequence.

The compositions and methods described herein can include the use of variants, homologs, and/or fragments of a reference SIAE nucleic acid, e.g., variants, homologs, and/or fragments of the SIAE nucleic acid sequence represented by SEQ ID NO:2. The terms “variant” or “homolog” in relation to SIAE nucleic acids include any substitution, variation, modification, replacement, deletion, or addition of one (or more) nucleotides from or to the sequence of a reference SIAE nucleic acid. The resultant nucleotide sequence may encode an SIAE polypeptide that is generally at least as biologically active or less biologically active as the referenced SIAE polypeptide (e.g., as represented by SEQ ID NO: 1). In particular, the term “homolog” covers homology with respect to structure and/or function provided that the resultant nucleotide sequence codes for or is capable of coding for an SIAE polypeptide being at least as biologically active as an SIAE encoded by a sequence shown herein as SEQ ID NO:1. With respect to sequence homology, there can be at least 75% (e.g., 85%, 90%, 95%, 98%, or 100%) homology to the sequence shown as SEQ ID NO: 2.

A “variant” of a protein is defined as an amino acid sequence which differs by one or more amino acids from a polypeptide sequence or any homolog of the polypeptide sequence. The variant may have conservative changes, wherein a substituted amino acid has similar structural or chemical properties, e.g., replacement of leucine with isoleucine. A variant may have nonconservative changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

Also included within the compositions and methods disclosed herein are certain alleles of the SIAE gene. As used herein, an “allele” or “allelic sequence” is an alternative form of SIAE. Alleles result from a mutation, i.e., a change in the nucleotide sequence, and generally produce altered mRNAs or polypeptides whose structure or function may or may not be altered. Any given gene can have none, one, or more than one allelic form. Common mutational changes that give rise to alleles are generally ascribed to deletions, additions, or substitutions of amino acids. Each of these types of changes can occur alone, or in combination with the others, one or more times in a given sequence. An SIAE allele or an SIAE allelic sequence contains one or more mutational changes as compared to SEQ ID NO: 2, or the reference human SIAE nucleic acid sequence. For example, an SIAE allele can have a mutation resulting in, for example, a full-length SIAE polypeptide containing one or more amino acid substitutions, e.g., one or more of the amino acid substitutions listed in Tables 3, 4 and 5, as compared to SEQ ID NO:1, the reference human SIAE amino acid sequence.

A nucleic acid encoding a mammalian, e.g., human, SIAE amino acid sequence can be amplified from human cDNA by conventional PCR techniques, using primers upstream and downstream of the coding sequence. SIAE polypeptides or fragments thereof can be produced and isolated using methods described herein.

The SIAE nucleic acids described herein include both RNA and DNA, including genomic DNA and synthetic (e.g., chemically synthesized) DNA. Nucleic acids can be double-stranded or single-stranded. Where single-stranded, the nucleic acid can be a sense strand or an antisense strand. Nucleic acids can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “isolated nucleic acid” means a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated SIAE nucleic acid includes some or all of the 5′ non-coding (e.g., promoter) sequences that are immediately contiguous to the SIAE nucleic acid coding sequence. The term includes, for example, recombinant DNA that is incorporated into a vector, into an autonomously replicating plasmid or virus, or into the genomic DNA of a prokaryote or eukaryote, or which exists as a separate molecule (e.g., a genomic DNA fragment produced by PCR or restriction endonuclease treatment) independent of other sequences. It also includes a recombinant DNA that is part of a hybrid gene encoding an additional polypeptide sequence.

The term “purified” refers to a nucleic acid or polypeptide that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

In some instances, the compositions and methods described herein can include the use of nucleic acid sequences that are substantially identical to an SIAE nucleic acid. A nucleic acid sequence that is “substantially identical” to an SIAE nucleic acid is at least 75% identical (e.g., at least about 80%, 85%, 90%, or 95% identical) to the SIAE nucleic acid sequences represented by SEQ ID NO: 2. For purposes of comparison of nucleic acids, the length of the reference nucleic acid sequence will typically be at least 50 nucleotides, but can be longer, e.g., at least 60 nucleotides, or more nucleotides.

To determine the percent identity of two amino acid or nucleic acid sequences, the sequences are aligned for optimal comparison purposes (i.e., gaps can be introduced as required in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of overlapping positions×100). The two sequences can be of the same length.

The percent identity or homology between two sequences is determined using the mathematical algorithm of Karlin and Altschul, Proc. Nat'l Acad. Sci. USA 87:2264-2268 (1990), modified as in Karlin and Altschul, Proc. Nat'l Acad. Sci. USA 90:5873-5877 (1993). Such an algorithm is incorporated into the NBLAST and XBLAST programs of Altschul, et al., J. Mol. Biol. 215:403-410 (1990). BLAST nucleotide searches are performed with the NBLAST program, score=100, wordlength=12 to obtain nucleotide sequences homologous to GR nucleic acid molecules of the invention. BLAST protein searches are performed with the XBLAST program, score=50, wordlength=3 to obtain amino acid sequences homologous to GR protein molecules of the invention. To obtain gapped alignments for comparison purposes, Gapped BLAST is utilized as described in Altschul et al., Nucleic Acids Res. 25:3389-3402 (1997). When utilizing BLAST and Gapped BLAST programs, the default parameters of the respective programs (e.g., XBLAST and NBLAST) are used. See online at ncbi.nlm.nih.gov.

The methods and compositions described herein can also include nucleic acids that hybridize, e.g., under stringent hybridization conditions (as defined herein) to all or a portion of the nucleotide sequences represented by SEQ ID NO: 2. The hybridizing portion of the hybridizing nucleic acids is typically at least 15 (e.g., 20, 30, or 50) nucleotides in length. The hybridizing portion of the hybridizing nucleic acid is at least about 75%, e.g., at least about 80%, 95%, 98% or 100%, identical to the sequence of a portion or all of a nucleic acid encoding an Siae polypeptide, or to its complement. Hybridizing nucleic acids of the type described herein can be used as a cloning probe, a primer (e.g., a PCR primer), or a diagnostic probe.

High stringency conditions are hybridizing at 68° C. in 5×SSC/5×Denhardt's solution/1.0% SDS, or in 0.5 M NaHPO₄ (pH 7.2)/1 mM EDTA/7% SDS, or in 50% formamide/0.25 M NaHPO₄ (pH 7.2)/0.25 M NaCl/1 mM EDTA/7% SDS; and washing in 0.2×SSC/0.1% SDS at room temperature or at 42° C., or in 0.1×SSC/0.1% SDS at 68° C., or in 40 mM NaHPO₄ (pH 7.2)/1 mM EDTA/5% SDS at 50° C., or in 40 mM NaHPO₄ (pH 7.2) 1 mM EDTA/1% SDS at 50° C. Stringent conditions include washing in 3×SSC at 42° C. The parameters of salt concentration and temperature can be varied to achieve the optimal level of identity between the probe and the target nucleic acid. Additional guidance regarding such conditions is available in the art, for example, by Sambrook et al., 1989, Molecular Cloning: A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The compositions and methods described herein can also include the use of genetic constructs (e.g., vectors and plasmids) that include an SIAE nucleic acid described herein, operably linked to a transcription and/or translation sequence to enable expression, e.g., expression vectors. A selected nucleic acid, e.g., a DNA molecule encoding an SIAE polypeptide, is operably linked to another nucleic acid molecule, e.g., a promoter, when it is positioned either adjacent to the other molecule or in the same or other location such that the other molecule can direct transcription and/or translation of the selected nucleic acid. These genetic constructs are useful for, e.g., the therapeutic and diagnostic methods described herein or testing the activity of an SIAE polypeptide.

Various engineered cells, e.g., transformed host cells, which contain an SIAE nucleic acid described herein, can be useful for the compositions and methods described herein. A transformed cell is a cell into which (or into an ancestor of which) has been introduced, by means of standard techniques, a nucleic acid encoding an SIAE polypeptide. Both prokaryotic and eukaryotic cells are included, e.g., mammalian cells (e.g., 293T cells), fungi (such as yeast), and bacteria (such as Escherichia coli), and the like.

III. Methods for Diagnosing Autoimmune Disorders

The disclosure provides methods of identifying and/or categorizing a subject who is suffering from or at risk of developing an autoimmune disorder by determining (1) whether the subject has a functionally defective SIAE allele or variant, e.g., an allele that encodes an SIAE polypeptide that lacks catalytic activity or is not secreted; (2) the level of 9-O-acetylated sialic acid on B cells in the subject; and/or (3) the serum level of SIAE in the subject. Treatment decisions may be based on the presence of an SIAE allele, the level of B cell acetylation and/or the serum level of SIAE in the subject.

The methods described herein are applicable in a wide variety of clinical contexts. For example, the methods can be used for diagnosing patients in hospitals and outpatient clinics, as well as the Emergency Department. The methods can be carried out on-site or in an off-site laboratory. One or more steps can be carried out by a third party.

The term “patient” or “subject” is used throughout the specification to describe an animal, human or non-human, rodent or non-rodent, to whom treatment or diagnosis according to the methods of the present invention is provided. Veterinary and non-veterinary applications are contemplated. The term includes, but is not limited to, birds, reptiles, amphibians, and mammals, e.g., humans, other primates, pigs, rodents such as mice and rats, rabbits, guinea pigs, hamsters, cows, horses, cats, dogs, sheep and goats. Typical patients or subjects include humans, farm animals, and domestic pets such as cats and dogs.

Detecting and Identifying SIAE Variants

Useful SIAE variants to identify in subjects in the methods described herein include those SIAE variants encoding SIAE polypeptides that have decreased functions, e.g., decreased catalytic activity or defective secretion. Evidence disclosed herein suggest that a defect in SIAE could result in the increased acetylation of α2-6-linked sialic acid on B cells and, thus, attenuate the ability of glycoproteins on B cells to ligate CD22 and generate inhibitory signals. Patients with these kinds of defective SIAE genes or proteins may have hyperactive B cell receptor (BCR) signaling, and therefore, may suffer from and/or be at risk of developing an autoimmune disorder. Indeed, data described herein demonstrate that there is enrichment of defective SIAE coding variants in autoimmune patients as compared to controls.

Methods are known in the art to detect the presence or absence of an SIAE variant or allele. For example, SIAE variants can be detected by sequencing exons, introns, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection (Schafer et al., Nat. Biotechnol. 15:33-39 (1995)), denaturing high performance liquid chromatography (DHPLC, Underhill et al., Genome Res. 7:996-1005 (1997)), infrared matrix-assisted laser desorption/ionization (IR-MALDI) mass spectrometry (WO 99/57318), and combinations of such methods.

Genomic DNA can be used in the analysis of SIAE variants. Genomic DNA typically is extracted from a biological sample such as a peripheral blood sample, but also can be extracted from other biological samples, including tissues (e.g., mucosal scrapings of the lining of the mouth or from renal or hepatic tissue). Standard methods can be used to extract genomic DNA from a blood or tissue sample, including, for example, phenol extraction. Alternatively, genomic DNA can be extracted with kits such as the Qiagen DNeasy Kit, the QIAamp® Tissue Kit (Qiagen, Valencia, Calif.), Wizard® Genomic DNA purification kit (Promega, Madison, Wis.) and the A.S.A.P.™ Genomic DNA isolation kit (Boehringer Mannheim, Indianapolis, Ind.).

cDNA can also be used in the analysis of SIAE alleles. Using conventional methods, cDNA can be synthesized from the mRNA fraction of the total RNA isolated from a biological sample.

An amplification step can be performed before proceeding with the detection method. For example, exons and/or introns of an SIAE gene can be amplified and then directly sequenced. Those of ordinary skill in the art would be able to design and make primers useful for amplification of portions of an SIAE gene using methods known in the art. High throughput automated (e.g., capillary or microchip based) sequencing apparati can be used. Nucleic acid analysis include sequencing with a pyrophosphate DNA sequencer (454 Life Sciences, New Haven, Conn.; see U.S. Pat. Pub. No. 20050130173) or optical sequencing (see, e.g., U.S. Pat. Pub. Nos. 20060024711, 20060136144, and 20060012793).

Mass spectroscopy (e.g., MALDI-TOF mass spectroscopy) can be used to detect nucleic acid mutations. In some cases (e.g., the MassEXTENDT™ assay, SEQUENOM, Inc.), selected nucleotide mixtures, missing at least one dNTP and including a single ddNTP is used to extend a primer that hybridizes near a mutation. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid mutations. For example, different terminator ddNTPs can be labeled with different fluorescent dyes. A primer can be annealed near or immediately adjacent to a mutation, and the nucleotide at the mutation site can be detected by the type (e.g., “color”) of the fluorescent dye that is incorporated.

Hybridization to microarrays can also be used to detect mutations. For example, a set of different oligonucleotides, with the mutant nucleotide at varying positions with the oligonucleotides can be positioned on a nucleic acid array. The extent of hybridization as a function of position and hybridization to oligonucleotides specific for the other allele can be used to determine whether a particular mutation is present. See, e.g., U.S. Pat. No. 6,066,454.

Hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

Allele specific hybridization also can be used to detect SIAE alleles, including complete haplotypes of a mammal. See, Stoneking et al., Am. J. Hum. Genet. 48:370-382 (1991); and Prince et al., Genome Res. 11:152-162 (2001). For example, samples of DNA or RNA from one or more patient can be amplified using pairs of primers and the resulting amplification products can be immobilized on a substrate (e.g., in discrete regions). Hybridization conditions can be selected such that a nucleic acid probe can specifically bind to the sequence of interest, e.g., the SIAE nucleic acid containing a particular SIAE allelic nucleotide sequence. Such hybridizations typically are performed under high stringency, as some allelic nucleotide sequences include only a single nucleotide difference. High stringency conditions can include, for example, the use of low ionic strength solutions and high temperatures for washing. For example, nucleic acid molecules can be hybridized at 42° C. in 2×SSC (0.3M NaCl/0.03 M sodium citrate/0.1% sodium dodecyl sulfate (SDS) and washed in 0.1×SSC (0.015M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some cases, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For SIAE allelic nucleotide sequences that introduce a restriction site, restriction digest with the particular restriction enzyme can differentiate the alleles. For SIAE allelic nucleotide sequences that do not alter a common restriction site, mutagenic primers can be designed that introduce a restriction site when the variant allele is present or when the wild type allele is present. A portion of an SIAE nucleic acid can be amplified using the mutagenic primer and a wild type primer, followed by digest with the appropriate restriction endonuclease.

Certain variants, such as insertions or deletions of one or more nucleotides, can change the size of the DNA fragment encompassing the allele. The insertion or deletion of nucleotides can be assessed by amplifying the region encompassing the allele and determining the size of the amplified products in comparison with size standards. For example, a region of an SIAE nucleic acid can be amplified using a primer set from either side of the allele. One of the primers can be labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when a particular allelic nucleic acid sequence is present or only when the wild type nucleic acid sequence is present (MSPCR or allele-specific PCR). For example, DNA from a patient and a control can be amplified separately using either a wild type SIAE primer or a primer specific for an SIAE allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type SIAE nucleic acid sequence would have amplification products only in the reaction using the wild type primer. Similarly, patient samples containing solely an SIAE allele would have amplification products only in the reaction using the allele-specific primer. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al., Genome 11:163-169 (2001).

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

The presence of an SIAE allele in a patient can also be detected by analyzing the SIAE polypeptide encoded by the SIAE allele. For example, the amino acid sequence encoded by an SIAE allele can be compared to a reference SIAE amino acid sequence (e.g., SEQ ID NO:1) using methods described herein and known in the art. Generally, a non-conservative amino acid substitution, in which an amino acid residue is replaced with an amino acid residue having a different kind of side chain, e.g., substitution of an amino acid with basic side chains for one with acidic side chains, can be more likely to result in a polypeptide with altered functions than conservative amino acid substitutions, e.g., substituting an amino acid with another with similar side chains.

An exemplary method for determining whether a patient has an SIAE variant can include drawing a sample, e.g., about 1 ml, of blood from the patient, then isolating genomic DNA from the blood sample using any methods described herein or known the art. A specific region (e.g., exon 1) of the SIAE gene can be amplified by PCR using a set of primers. The amplified PCR product can be purified, cloned into plasmids, and sequenced to determine whether the patient has an SIAE allele with one or more mutations as compared to an SIAE reference nucleic acid sequence (e.g., SEQ ID NO:2). Those skilled in the art can appreciate that any combination of a forward primer and a reverse primer for a particular exon of the SIAE gene (e.g., the primers listed in Table 3) can be used to amplify the exon to determine whether that exon contains one or more mutations.

The amino acid sequences encoded by alleles with one or more mutations can be compared to an SIAE reference amino acid sequence (e.g., SEQ ID NO:1). An SIAE polypeptide with one or more mutations as compared to the reference SIAE amino acid sequence can be further tested to determine whether the polypeptide is functional, e.g., whether the SIAE polypeptide has an activity. SIAE activities can include, but are not limited to, ability to catalyze an esterase reaction, ability to be secreted out of cells and ability to mediate B cell receptor signaling. Skilled practitioners will appreciate that conventional assays, including those assays described herein, can be used to determine whether an SIAE polypeptide exhibits any of these activities. For example, C-terminal Flag-tagged human SIAE cDNAs cloned from cells (e.g., MDA-MB 231 cells) containing the changes corresponding to the coding variants (e.g., via conventional site-directed mutagenesis techniques) can be created. Each cDNA can be transfected into host cells (e.g., 293T cells). Lysate and supernatant can be collected. SIAE can be immunoprecipitated from the lysate and supernatant with, e.g., anti-Flag antibodies. Quantitative Western blot assays can be used to determine whether the SIAE is secreted. Esterase assays, e.g., one that uses a fluorogenic substrate, 4-methylumbelliferyl acetate, can be used to test whether the SIAE is catalytically active.

Certain SIAE variants (e.g., those described below) can produce SIAE polypeptides having decreased esterase activity (e.g., 50% or less as compared to the reference SIAE polypeptide). Other SIAE alleles described herein contain mutations that result in SIAE polypeptides that have a defect in secretion. Some SIAE variants may result in SIAE polypeptides that are both catalytically defective and poorly secreted.

Patients found to have one or more alleles of the SIAE gene that encode functionally defective SIAEs may be at risk of developing an autoimmune disorder. A patient suffering from an autoimmune disorder who is found to have one or more defective alleles of the SIAE gene may be candidates to be rationally treated with therapies directed to address this underlying defect.

In some instances, it may be useful to determine whether a particular SIAE allele is dominantly negative. For example, data disclosed below demonstrate that certain variants that result in SIAE polypeptides that are both catalytically defective and poorly secreted are dominantly negative. If an allele is not dominantly negative, it is expected that only patients homozygous for the allele may be considered to be at risk of developing autoimmunity. Whether a variant is dominantly negative can be determined using methods described herein and methods known in the art.

Determining Level of 9-O-Acetylation of Sialic Acid on B Cells

Data described herein demonstrate that 9-O-acetylation of sialic acid was increased in B cells from Siae^{Δ2/Δ2} mice as compared with wildtype B cells. Evidence also suggest that some autoimmune patients (e.g., SLE patients) may exhibit increased 9-O-acetylation of sialic acid on B cells, presumably because these patients have functionally defective SIAE (e.g., due to one or more alleles of the SIAE gene that encode defective SIAE polypeptides). Therefore, quantifying the amount of 9-O-acetyl sialic acid on B cells from human patients can be used to determine (1) whether a patient who has been diagnosed with an autoimmune disorder has defective SIAE, and if so, to identify appropriate treatments (e.g., a treatment method described herein comprising administering SIAE protein); and (2) whether a patient is at risk of or predisposed to developing an autoimmune disorder.

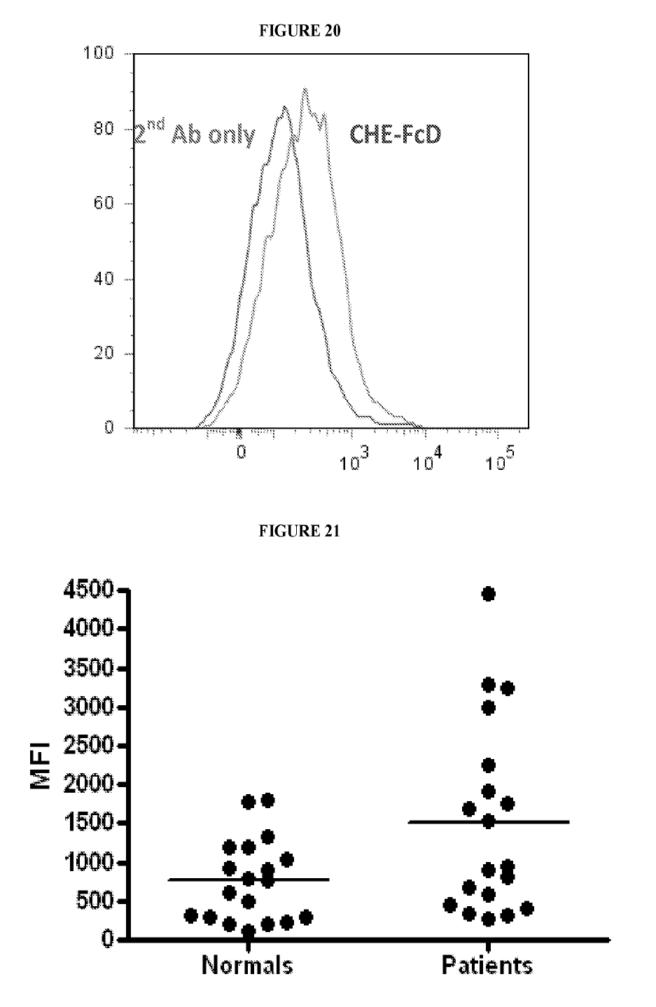

The level of 9-O-acetylated sialic acids on B cells can be quantified using methods described herein and known in the art. For example, flow cytometry-based methods using a detection agent such as the CHE-FcD probe can be used. The CHE-FcD probe, a fusion protein composed of the extracellular domains of the influenza C hemagglutinin esterase (CHE) which binds 9-O-acetylated sialic acids, and the Fc portion of human IgG 1 (Fc), treated with diisopropylfluorophosphate (D) can be generated as previously described (Krishna and Varki, J Exp Med 185:1997-2013 (1997); Martin et al., Methods Enzymol 363: 489-498 (2003)). Other probes comprising proteins that can bind to 9-O-acetylated sialic acids can also be generated and used in the diagnostic methods described herein. For example, an O-acetyl esterase from coronavirus with the active site serine residue mutated can be used to generate a probe to detect 9-O-acetylated sialic acids on B cells. The probe, e.g., CHE Fc-D protein, can be precomplexed with a fluorescent dye-anti-human IgG, e.g., Cy5 or FITC-F(ab′)₂ goat anti-human IgG, or IRDye 800CW-anti human IgG. Human B cells can then be added to the precomplex. The cells can the be washed, reacted with, e.g., 2.4G2, an Fcγ III/II receptor-blocking antibody, and surface stained using methods known in the art. Conventional flow cytometric analysis can then be perform to detect 9-O-acetyl sialic acid on the human B cells. Flow cytometric analysis showing a shift to the right (see, FIG. 20) when using CHE-Fc-D and the labeled second antibody as compared with the second antibody alone indicates that the B cells have 9-O-acetylated sialic acids. The degree of shift can be quantitated, e.g., as mean fluorescence intensity, using methods known in the art. Other methods known in the art can also be used to detect and quantify 9-O-acetyl sialic acid on the human B cells.

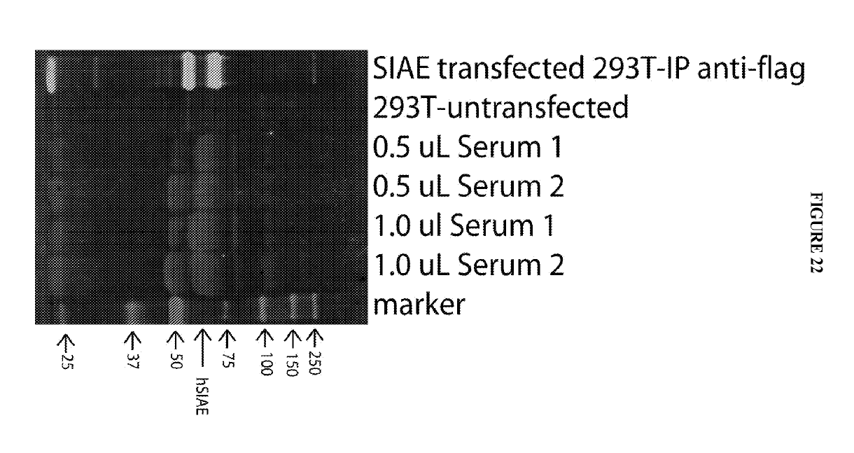

A normal or threshold level (e.g., a level found in normal subjects) of 9-O-acetylated sialic acid on B cells can be the mean level (e.g., expressed as mean fluorescence intensity (MFI)) found in a large group of control or normal subjects. Conventional statistical analytical methods can be used to determine a normal or threshold MFI. A patient found to have a higher level of 9-O-acetylated sialic acid on B cells as compared to a control level may be considered to be at risk of developing an autoimmune disorder, or has a defect in an SIAE function. For example, an MFI that is one standard deviation greater than the normal or control mean MFI can be considered to indicate the presence of an elevated or abnormal level of 9-O-acetylated sialic acids on B cells. See, e.g., FIG. 21.

B cells from a human patient can be isolated using conventional methods known in the art. For example, a blood sample, e.g., 15-20 ml, can be collected from a patient, and B cells can be purified from the sample using RosetteSep (STEMCELL Technologies, Canada). The purified B cells can be analyzed immediately or frozen (e.g., in fetal calf serum and DMSO) for later analysis.

High throughput and/or automated methods, e.g., using multi-well plates, can be used to carry out the diagnostic methods described herein. For example, B cells from each patient can be dispensed manually or by a robot into a well of a multi-well plate, e.g., a 96-well or 384-well plate, and the cells can be incubated with premixed conjugates of CHE-FcD and a fluorescent dye-anti human IgG. Fluorescence can then be quantified per well using imaging systems known in the art, for example, LiCor Aerius (Li-Cor Biosciences).

Determining Serum/Plasma Level of SIAE

Data disclosed herein show that SIAE is a secreted protein and can be detected in the serum of human subjects, and therefore, has the potential to remove acetyl groups from 9-O-acetylated Siglec ligands at the cell surface. The application also discloses evidence showing that some alleles of the SIAE gene found in autoimmune patients encode SIAEs that have defects in secretion. Without wishing to be bound by theory, increased acetylation of α2-6-linked sialic acid on B cells may attenuate the ability of glycoproteins on B cells to ligate CD22 and lead to hyperactive B cell signaling. This hyperactive B cell signaling may lead to autoimmunity, as evidence described herein suggests. Thus, a subject with a low serum level of SIAE (e.g., because of a defect in secretion or expression of SIAE) as compared to a normal subject may be predisposed to autoimmunity. A low serum level of SIAE in a patient suffering from an autoimmune disorder suggests that a defect in SIAE may be an underlying cause of the disorder.

The level of SIAE in the serum or plasma of a patient can be quantified using conventional methods. For example, polyclonal and monoclonal antibodies (or fragments thereof) that specifically bind to an SIAE polypeptide can be used to detect SIAE in conventional immunoassays of biological tissues or extracts. Examples of suitable assays include, without limitation, Western blotting, ELISAs, radioimmune assays, and the like. Antibodies that specifically bind to an SIAE polypeptide can be generated using conventional methods known in the art and described herein. Commercially available antibodies to SIAE (e.g., from Abcam (Cambridge, Mass.)) can also be used in the methods described herein.

A normal or threshold level of human serum SIAE can be the level found in a normal subject or the mean level found in a large group of normal subjects (e.g., 100 or more subjects). For example, about 10-30 mg/ml of SIAE in human serum may be considered as a normal level of serum SIAE. A patient having a serum SIAE level that is less than the normal level may indicate that the patient is at risk for developing an autoimmune disorder or has a defect in an SIAE function. For example, a serum SIAE level that is at least 30% (e.g., 33%, 45% and 55%) less than a normal level may be considered as abnormal.

An antibody “specifically binds” to a particular antigen, e.g., an SIAE polypeptide, when it binds to that antigen, and binds to a significantly lesser extent (e.g., with significantly lower affinity or not at all) to other molecules in a sample, e.g., a biological sample that includes an SIAE polypeptide. The antibodies described herein include monoclonal antibodies, polyclonal antibodies, humanized or chimeric antibodies, monospecific antibodies, single chain antibodies, Fab fragments, F(ab′)₂ fragments, and molecules produced using a Fab expression library.

As used herein, the term “antibody” refers to a protein comprising at least one, e.g., two, heavy (H) chain variable regions (abbreviated herein as VH), and at least one, e.g., two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al., J. Mol. Biol. 196:901-917 (1987)). Each VH and VL is composed of three CDR's and four FRs, arranged from amino-terminus to carboxy-terminus in the following order: FR1, CDR1, FR2, CDR2, FR3, CDR3, FR4.

An anti-SIAE antibody can further include a heavy and light chain constant region, to thereby form a heavy and light immunoglobulin chain, respectively. The antibody can be a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2, and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

An “SIAE binding fragment” of an antibody refers to one or more fragments of a full-length antibody that retain the ability to specifically bind to an SIAE polypeptide or a portion thereof. Examples of SIAE polypeptide binding fragments of an anti-SIAE antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 242:423-426 (1988); and Huston et al., Proc. Natl. Acad. Sci. USA 85:5879-5883 (1988)). Such single chain antibodies are also encompassed within the term “SIAE binding fragment” of an antibody. These antibody fragments can be obtained using conventional techniques known to those with skill in the art.

To produce antibodies, SIAE polypeptides (or antigenic fragments (e.g., fragments of SIAE that appear likely to be antigenic by criteria such as high frequency of charged residues) or analogs of such polypeptides), e.g., those produced by standard recombinant or peptide synthetic techniques (see, e.g., Ausubel et al., supra), can be used. In general, the polypeptides can be coupled to a carrier protein, such as KLH, as described in Ausubel et al., supra, mixed with an adjuvant, and injected into a host mammal. A “carrier” is a substance that confers stability on, and/or aids or enhances the transport or immunogenicity of, an associated molecule. For example, nucleic acids encoding SIAE or fragments thereof can be generated using standard techniques of PCR, and can be cloned into a pGEX expression vector (Ausubel et al., supra). Fusion proteins can be expressed in E. coli and purified using a glutathione agarose affinity matrix as described in Ausubel, et al., supra.

Typically, to produce antibodies, various host animals are injected with SIAE polypeptides. Examples of suitable host animals include rabbits, mice, guinea pigs, rats, and fowl. Various adjuvants can be used to increase the immunological response, depending on the host species, including but not limited to Freund's (complete and incomplete adjuvant), adjuvant mineral gels such as aluminum hydroxide, surface active substances such as lysolecithin, pluronic polyols, polyanions, peptides, oil emulsions, keyhole limpet hemocyanin, dinitrophenol, BCG (bacille Calmette-Guerin) and Corynebacterium parvum. Such procedures result in the production of polyclonal antibodies, i.e., heterogeneous populations of antibody molecules derived from the sera of the immunized animals. Antibodies can be purified from blood obtained from the host animal, for example, by affinity chromatography methods in which the SIAE polypeptide antigen is immobilized on a resin.

Monoclonal antibodies (mAbs), which are homogeneous populations of antibodies to a particular antigen, can be prepared using SIAE polypeptides and standard hybridoma technology (see, e.g., Kohler et al., Nature, 256:495 (1975); Kohler et al., Eur. J. Immunol., 6:511 (1976); Kohler et al., Eur. J. Immunol., 6:292 (1976); Hammerling et al., In Monoclonal Antibodies and T Cell Hybridomas, Elsevier, N.Y. (1981); Ausubel et al., supra).

Typically, monoclonal antibodies are produced using any technique that provides for the production of antibody molecules by continuous cell lines in culture, such as those described in Kohler et al., Nature, 256:495 (1975), and U.S. Pat. No. 4,376,110; the human B-cell hybridoma technique (Kosbor et al., Immunology Today, 4:72 (1983); Cole et al., Proc. Natl. Acad. Sci. USA, 80:2026, (1983)); and the EBV-hybridoma technique (Cole et al., Monoclonal Antibodies and Cancer Therapy, Alan R. Liss, Inc., pp. 77-96, (1983)). Such antibodies can be of any immunoglobulin class including IgG, IgM, IgE, IgA, IgD, and any subclass thereof. The hybridomas producing the mAbs of this invention can be cultivated in vitro or in vivo.

Once produced, polyclonal or monoclonal antibodies can be tested for recognition, e.g., specific recognition, of SIAE in an immunoassay, such as a Western blot or immunoprecipitation analysis using standard techniques, e.g., as described in Ausubel et al., supra. Antibodies that specifically bind to an SIAE polypeptide, or conservative variants thereof, are useful in the invention. For example, such antibodies can be used in an immunoassay to detect an SIAE polypeptide in a sample, e.g., plasma or serum.

Alternatively or in addition, an antibody can be produced recombinantly, e.g., produced by phage display or by combinatorial methods as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. Bio/Technology 9:1370-1372 (1991); Hay et al., Hum Antibod Hybridomas 3:81-85 (1992); Huse et al., Science 246:1275-1281 (1989); Griffths et al., EMBO J. 12:725-734 (1993); Hawkins et al., J Mol Biol 226:889-896 (1992); Clackson et al., Nature 352:624-628 (1991); Gram et al., PNAS 89:3576-3580 (1992); Garrad et al., Bio/Technology 9:1373-1377 (1991); Hoogenboom et al., Nuc Acid Res 19:4133-4137 (1991); and Barbas et al., PNAS 88:7978-7982 (1991).

Anti-SIAE antibodies can be fully human antibodies (e.g., an antibody made in a mouse which has been genetically engineered to produce an antibody from a human immunoglobulin sequence), or non-human antibodies, e.g., rodent (mouse or rat), goat, primate (e.g., monkey), camel, donkey, porcine, or fowl antibodies.

An anti-SIAE antibody can be one in which the variable region, or a portion thereof, e.g., the CDRs, are generated in a non-human organism, e.g., a rat or mouse. The anti-SIAE polypeptide antibody can also be, for example, chimeric, CDR-grafted, or humanized antibodies. The anti-SIAE polypeptide antibody can also be generated in a non-human organism, e.g., a rat or mouse, and then modified, e.g., in the variable framework or constant region, to decrease antigenicity in a human.

Techniques developed for the production of “chimeric antibodies” (Morrison et al., Proc. Natl. Acad. Sci., 81:6851, 1984; Neuberger et al., Nature, 312:604, 1984; Takeda et al., Nature, 314:452, 1984) can be used to splice the genes from a mouse antibody molecule of appropriate antigen specificity together with genes from a human antibody molecule of appropriate biological activity. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; and U.S. Pat. Nos. 4,946,778 and 4,704,692) can be adapted to produce single chain antibodies against an SIAE polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments can include but are not limited to F(ab′)₂ fragments, which can be produced by pepsin digestion of the antibody molecule, and Fab fragments, which can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

IV. Method for Treating Autoimmune Disorders

As described herein, adding purified catalytically active SIAE to murine Siae mutant B cells in vitro can rescued the enhanced B cell receptor (BCR) signaling exhibited by the cells. Functionalized sialic acid has been used to treat mouse erythro-leukemia cell lines (Shi et al., The Journal of Biological Chemistry 271: 31517-31525 (1996), as well as in a diagnostic assay for childhood acute leukemia (Ghosh et al., Journal of Cellular Biochemistry 95: 206-216 (2005)). Further, sialylated proteins have previously been investigated for the treatment of immune disorders as disclosed in U.S. Pat. No. 5,858,969.

Disclosed herein are methods of treating a patient suffering from an autoimmune disorder by administering a catalytically active SIAE polypeptide or functional fragment thereof to a patient suffering from an autoimmune disorder, e.g., as described herein. A patient who is found to have defective SIAE (e.g., as determined by the diagnostic methods described herein) would be particularly suitable for this treatment.

SIAE polypeptides (e.g., naturally occurring, recombinant and fusion proteins) or functional fragments thereof useful for the treatment methods described herein can be generated and purified using methods known in the art and described herein. For example, human and/or murine SIAE polypeptides can be expressed expressed and purified from a stably transfected human cell line (e.g., flag tagged human or murine SIAE in U2OS cell line). Conventional methods, e.g., affinity chromatography, can be used to purify SIAE proteins.

Pharmaceutical Compositions and Methods of Administration

Included herein are pharmaceutical compositions (e.g., comprising a protein and/or enzyme as described above; i.e., for example, an SIAE enzyme or enzyme fragment) for the treatment of an autoimmune disease. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models (e.g., the Siae^{Δ2/Δ2} mice described herein) or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g per kg of body weight, e.g., once or more daily or yearly.

In one embodiment, the present invention contemplates delivering a pharmaceutical composition within a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

In one embodiment, the present invention contemplates liposomes capable of attaching and releasing SIAE proteins, protein fragments, and/or oligonucleotides. Liposomes are microscopic spherical lipid bilayers surrounding an aqueous core that are made from amphiphilic molecules such as phospholipids. Water soluble compounds can be entrapped in the core and lipid-soluble compounds can be dissolved in the shell-like bilayer.

Liposomes have a special characteristic in that they enable water soluble and water insoluble chemicals to be used together in a medium without the use of surfactants or other emulsifiers. For example, liposomes form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

The present invention contemplates cationic and anionic liposomes, as well as liposomes having neutral lipids comprising SIAE proteins, protein fragments, and oligonucleotides. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

A medium comprising liposomes that provide controlled release of SIAE proteins, protein fragments or oligonucleotides can be used in the methods described herein. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes may be broadly categorized into two classifications. Conventional liposomes are generally mixtures of stabilized natural lecithin (PC) that may comprise synthetic identical-chain phospholipids that may or may not contain glycolipids.

Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. East Setauket, N.Y.) is known to manufacture custom designed liposomes for specific delivery requirements.

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel. It is intended that the terms “microspheres, microcapsules and microparticles” (i.e., measured in terms of micrometers) are synonymous with their respective counterparts “nanospheres,” “nanocapsules” and “nanoparticles” (i.e., measured in terms of nanometers). Further, the terms “micro/nanosphere,” “micro/nanocapsule” and “micro/nanoparticle” are used interchangeably, as discussed herein.

Microspheres may be obtainable commercially (i.e., for example, PROLEASE®, Alkermes (Cambridge, Mass.)). For example, a freeze dried SIAE protein medium is homogenized in a suitable solvent and sprayed to manufacture microspheres in the range of 20 to 90 μm. Techniques are then followed that maintain sustained release integrity during phases of purification, encapsulation and storage. See Scott et al., Nature Biotechnology, Volume 16:153-157 (1998).

Modification of the microsphere composition by the use of biodegradable polymers can provide an ability to control the rate of SIAE protein, protein fragment, or oligonucleotide release. Miller et al., J. Biomed. Mater. Res., Vol. 11:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of SIAE protein is added to the biodegradable polymer metal salt solution. The weight ratio of SIAE protein to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and SIAE protein is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer and SIAE protein mixture are: i) phase separation during a gradual addition of a coacervating agent; ii) an in-water drying method or phase separation method, where an antiflocculant is added to prevent particle agglomeration and iii) by a spray-drying method.

The methods described herein can include the use of a medium comprising a microsphere or microcapsule capable of delivering a controlled release of a compound for a duration of approximately, e.g., between 1 day and 6 months. Controlled release microcapsules may be produced by using known encapsulation techniques such as centrifugal extrusion, pan coating and air suspension. Many techniques to produce such microspheres/microcapsules can be engineered to achieve particular release rates. For example, OLIOSPHERE® (Macromed) is a controlled release microsphere system. These particular microsphere's are available in uniform sizes ranging between 5-500 μm and composed of biocompatible and biodegradable polymers. Specific polymer compositions of a microsphere control the drug release rate such that custom-designed microspheres are possible, including effective management of the burst effect. PROMAXX® (Epic Therapeutics, Inc.) is a protein-matrix drug delivery system. The system is aqueous in nature and is adaptable to standard pharmaceutical drug delivery models. In particular, PROMAXX® is a bioerodible protein microsphere that can deliver both small and macromolecular compounds, and may be customized regarding both microsphere size and desired release characteristics.

A microsphere or microparticle can comprise a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. See, e.g., Lew C. W., Controlled-Release pH Sensitive Capsule And Adhesive System And Method. U.S. Pat. No. 5,364,634 (herein incorporated by reference).

A microparticle can comprise a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a compound (i.e., for example, an SIAE protein) is directly bound to the surface of the microparticle or is indirectly attached using a bridge or spacer. The amino groups of the gelatin lysine residues are easily derivable to provide sites for direct coupling of a compound. Alternatively, spacers (i.e., linking molecules and dramatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

The terms “effective amount” and “effective to treat,” as used herein, refer to an amount or a concentration of a compound utilized for a period of time (including acute or chronic administration and periodic or continuous administration) that is effective within the context of its administration for causing an intended effect or physiological outcome. Effective amounts of compound for use in the present treatment methods include, for example, amounts that, e.g., modulate BCR signaling, decreases level of 9-O-acetylated sialic acid on B cells, or generally improve the prognosis of a patient diagnosed with an autoimmune disorder. The term “treat(ment)” is used herein to describe delaying the onset of, inhibiting, or alleviating the detrimental effects of a condition, e.g., an autoimmune disorder.

### EXAMPLES

### Example 1

### Generation of Homozygous SIAE^{Δ2/Δ2} Mice and Elucidation of the Function of SIAE In Vivo

### Materials and Methods

### Generation of Homozygous SIAE^{Δ2/Δ2} Mice

In 8-12 week old C57BL/6 and B6.129S7-Ragl mice (Jackson Laboratories, Bar Harbor, Me.) exon 2 of SIAE encoding the N-terminus of the matured SIAE small chain was deleted. The mice used in these studies were backcrossed into the C57BL/6 background for ten generations and then maintained by intercrossing. The Siae targeting vector was assembled from a 129/Sv genomic clone by inserting the 0.7-kb BamHI-XbaI fragment containing exon 2 of Siae, into the BamHI site of the pflox vector as described33. Adjacent 129/Sv Siae genomic sequences were added by subcloning the 1.5-kb XbaI-NheI fragment into the XhoI site and the BamHI-BamHI 9 kb fragment into the SalI site of pflox, respectively. Ten micrograms of NotI-linearized targeting vector was electroporated into R1 embryonic stem (ES) cells and G418 resistant transfectants (150 μg/ml), positive by PCR for homologous recombination and retaining all three loxP sites were transfected with the pCreHygro expression vector. Following 4 days of gancyclovir (2 μM) selection, subclones were isolated and those harboring the Siae-floxed allele were detected by Southern blotting with BamHI-SpeI genomic digestion and a loxP probe. The B3 and B9 ES cell clones were used to generate chimeric mice in C57BL/6 host embryos. Offspring were genotyped by Southern blotting with BamHI-SpeI tail DNA hybridized to the 3′ probe (3S0.6). Heterozygous offspring were mated to C57BL/6 ZP3-Cre mice. Female off spring were then mated to produce homozygous SiaeΔ2/Δ2 mice. PCR primers used for genotyping were as follows: PS-LSEG-16, 5′ TTTAGGAGCAAG GGGTTGGCCAAGA 3′ (SEQ ID NO:3); PS-LSE-B: 5′GGTTTCCTGACCTTGGCACAAGGT 3′ (SEQ ID NO:4); and LOX-R: 5′ CGGTACCCGGGGATCAATTCGAG 3′ (SEQ ID NO:5). PS-LseG-16 and PS-LseG-B yield the 420 bps WT band and PS-LseG-16 and LOX-R the 340 bps mutant band.

### Antibodies, Staining and Flow Cytometry (FCM)

The following murine monoclonal antibody conjugates were used: Allophycocyanin (APC)-RA3-6B2 (anti-CD45R/B220, rat IgG2a, κ), fluorescein isothiocyanate (FITC)-S7 (anti-CD43, rat IgG2a, κ), FITC-7G6, (anti-CD21/CD35, rat IgG2b), FITC-Cy34.1 (anti-CD22.2, mouse (DBA/1) IgG 1, κ, APC-53-7.3 (anti-CD5, rat IgG2a, κ), APC-M1/70 (anti-CD11b, rat IgG2b, κ) r-phycoerythrin (R-PE)-GK1.5 (anti-CD4, rat IgG2b, κ), and FITC-53-6.7 (anti-CD8a, rat IgG2a, κ) all from BD-Pharmingen, San Jose, Calif.; APC and R-PE-1B4B1 (anti-IgM, rat IgG), and R-PE-11-26 (anti-IgD, rat IgG2a, κ) from Southern Biotech, Birmingham, Ala.).

Single cell suspensions were made from spleen, bone-marrow (one femur and tibia), thymus and peritoneal washings using standard methodology. Red cells were lysed with 2 ml of ACK lysing buffer (Cambrex, Walkersville, Md.). The lysis buffer was neutralized by adding 10 ml of PBS-0.2% BSA (PBA). Prior to staining, 1×10⁶ cells were reacted with 2.5 μg of 2.4G2 (anti-CD16/CD32 {FcγIII/II receptor}, rat IgG2b, k, BDPharmingen). Surface staining was performed using appropriate dilutions of antibodies in 12×75 mm round-bottom polystyrene tubes in a volume of 200 μl for 30 minutes (min) in the dark at 4° C. Flow cytometric analysis was performed as previously described (Cariappa et al., 1999. J. Immunol. 162 4417-4423) on a dual laser FC500 (Beckman Coulter Corp., Miami, Fla.), a MoFlo (DakoCytomation, Fort Collins, Colo.) and a FACSAria (Beckton, Dickinson, and Company, San Jose, Calif.). Unstained cells were used to set voltage and single color positive controls were used for electronic compensation. Viable cells were determined by forward and side scatter characteristics and 3×10⁴ to 5×10⁴ gated events were collected. Gates were set as previously described (Cariappa et al., 2007. Blood. 109: 2339-2345). Processed samples were analyzed using and RXP (Beckman Coulter), and FloJo v8.4.1 (Treestar Inc.) analysis software.

### Reconstitution of the Lymphoid Compartment in Rag-1 Deficient Mice

Rag-1^{-/-} mice were irradiated with 9.5 Gy and reconstituted with 5×10⁶ wildtype or mutant adult bone marrow cells via tail vein injection. Reconstitution of the bone marrow and spleen was assessed 7 weeks later by FCM.

### Detection of 9-O-Acetylated Sialic Acid on Splenocytes

The CHE-FcD probe, a fusion protein composed of the extracellular domains of the influenza C hemagglutinin esterase (CHE) which binds 9-O-acetylated sialic acids, and the Fc portion of human IgG 1 (Fc), treated with diisopropylfluorophosphate (D) was generated as previously described (Krishna and Varki, J Exp Med 185: 1997-2013 (1997); Martin et al., Methods Enzymol 363: 489-498 (2003)). The chimeric CHE Fc-D protein was precomplexed with Cy5 or FITC-F(ab′)₂ goat anti-human IgG (Jackson ImmunoResearch Laboratories; 1 μl of a 1:10 dilution of CHE-FcD in PBA with 2 μl of the secondary antibody in a total volume of 50 μl PBA) for 2 hours (h) at 4° C. in the dark. 10⁶ cells in 50 μl PBA were preincubated for 45 min at 37° C., added to the precomplex, and incubated on ice for an additional 1.5 h. The cells were washed once, reacted with 2.4G2, an Fcγ receptor-blocking antibody, and surface stained as described earlier. Staining of acetylated sialic acid on CD4+T cells served as a positive control (Krishna and Varki, J Exp Med 185: 1997-2013 (1997)).

### B Cell Receptor Crosslinking and Accumulation of Cytosolic Calcium

Briefly, Indo-1 AM (acetoxymethyl ester, Molecular Probes, Eugene, Oreg.) was added to 3×10⁶ splenocytes in 500 μl of Hanks Balanced Salt Solution (HBSS, containing calcium chloride, magnesium chloride and magnesium sulfate, GIBCO-Invitrogen, Carlsbad, Calif.)-10% fetal calf serum (HBSS-10). The final concentration of Indo-1 was 1 μM. The cells were incubated in a 37° C. waterbath for 30 min, equilibrated to RT for 5 min and washed with HBSS containing 2 mM Hepes buffer and no serum (HBSH-0), all manipulations including the incubation were in the dark. Cells were then surface stained as described earlier and washed with HBSH-0, all in the dark at 4° C. The cells were resuspended in the residual volume and an additional 50 μl of HBSH-0 was added to make a total volume of approximately 150 μl. The cells were then split into three 50 μl aliquots and the volume made up to 100 μl with HBSH-0. The cells were held in the dark at 4° C. prior to analysis. Prior to calcium flux measurements, the cells were filtered and equilibrated to RT for 10 min. The cells were warmed to 37° C. in a heat block for 2 min, and placed on the flow cytometer in a chamber maintained at 37° C. After a flow rate of approximately 500 cells/sec was established, acquisition was begun to record a 30 seconds (s) base line. Following addition of stimulatory antibody acquisition was continued for another 180 s with the cells at 37° C. The flow cytometer used was a FACS Vantage SE (Beckton Dickinson and Company) operating with a Coherent Innova Enterprise LASER (Model 621).

### Serum Immunoglobulins and Autoantibodies

Total serum immunoglobulins of various isotypes were analyzed using an ELISA approach (Alpha Diagnostic International, San Antonio, Tex.). ELISA was also used to quantify serum autoantibodies (anti-dsDNA, anti-ssDNA, anti-histone, anti-Sm) and circulating immune complexes (all from Alpha Diagnostic).

### Immunofluorescence Staining

Immunofluorescence was performed as previously described with the some modifications. Sections for immunofluorescence were blocked with 20% normal goat serum in 1% BSA/1×PBS for 20 min and without washing stained with FITC-AffiniPure F(ab′)₂ goat anti-mouse IgG (H+ L, Jackson Immunoresearch) diluted 1:200 in 5% normal goat serum/1% BSA/PBS for 15′ in the dark at room temperature. After rinsing in PBS, thrice for 5′ each, sections were mounted using 25 ul of Vectashield (Vector Laboratories, Burlingame, Calif.). Immunohistochemistry was performed as described previously.

### Immunoprecipitation and Western Blot Analysis (CD22/SHP-1)

The method of Poe et al. (2004, Nat. Immunol. 5:1078-1087) was followed with some modifications. Murine splenocytes (1×10⁷) or sorted B cells (5×10⁵) were lysed using 1% NP40 in PBS. Lysates were immunoprecipitated using anti-CD22 (CD-22, rabbit IgG, Epitomics, Burlingame, Calif.) and protein A Sepharose, and following separation on an SDS/polyacrylamide gel and Western transfer, membranes were probed with antibodies to CD22, SHP-1 and phosphotyrosine (anti-CD22.2, clone Lyb-8.2, BD-Pharmingen, anti-SHP1/2 rabbit polyclonal IgG, clone NL213, and anti-phosphotyrosine clone 4G10, Upstate-Millipore).

### Sequence Analysis of SIAE

An annealing temperature of 58° C. was used. Purification of the amplified products, bidirectional automated sequencing and analysis of the chromatograms were performed as described earlier.

### CFSE Staining and B Cell Proliferation

Analysis of CFSE stained cells was performed on non-stimulated and anti-IgM activated B cells as described earlier (Cariappa et al., 2003. J. Immunol. 171:1875-1880) with the following modification: CFSE (Molecular Probes) was added to a final concentration of 1 μM and no additional surface staining of the B cells was performed.

Immunization with T-Dependent and T-Independent Antigens and ELISA for Anti-DNP Antibodies

Mice were immunized i.p., with 100 μg of Di-nitrophenol (DNP) or DNP-Ficoll (Biosearch Technologies Inc., Novato, Calif.) in 1×PBS on day 0 and boosted on day 28. Serum was collected on designated days and antibodies were quantitated by ELISA.

### Results

### SIAE is Secreted and Can Access the Cell Surface

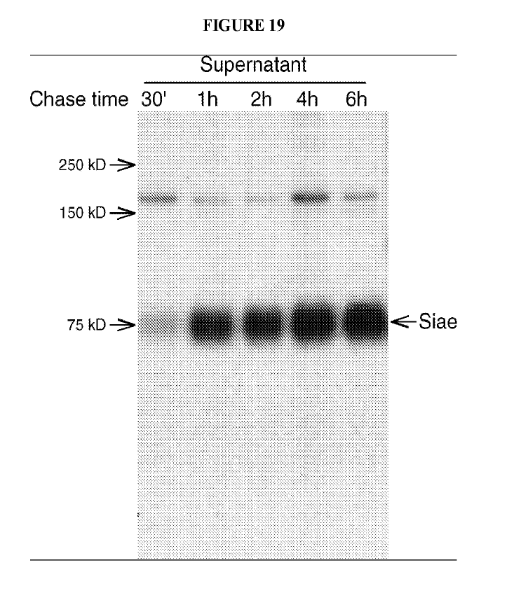

The Siae gene can generate two alternatively spliced variants. One form contains a signal peptide and can encode a protein that enters the secretory pathway (originally called Lse [lysosomal sialic acid acetylesterase]), whereas the other lacks this signal peptide and encodes a cytosolic esterase (Guimaraes et al., 1996. J. Biol. Chem. 271: 13697-13705; Stoddart et al., 1996. Nucleic Acids Res. 24: 4003-4008; Takematsu et al., 1999. J. Biol. Chem. 274 25623-25631). Lse protein was previously thought to localize to lysosomes. U2OS-Siae cells (U2OS cells stably transfected with a murine C-terminal Flag-tagged Siae cDNA in the pcDNA3.1 vector) were pulsed with 0.5 mCi of 35[S]methionine for 10 min and supernatants were immunoprecipitated immediately after labeling and at various chase time periods. Flag-tagged Siae was identified in cell lysates and in supernatants by immunoprecipitation with anti-Flag antibodies followed by separation on SDS/PAGE and autofluorography (FIG. 19). U2OS-Siae cells were permeabilized and stained with anti-Flag antibodies, wheat germ agglutinin, or antibodies to LAMP-1, and it was shown that Siae was expressed on the surface of transfected U2OS cells and partially in lysosomes (data not shown). Thus, it was demonstrated that, in transfected cells, this protein exhibited, at best, a limited lysosomal localization and was secreted and could bind to the surface of transfected cells. The enzyme, therefore, has the potential to remove acetyl groups from 9-O-acetylated Siglec ligands in a post Golgi vesicle or at the cell surface.

### Enhanced B Lymphocyte Antigen Receptor Signaling in Siae^{Δ2/Δ2} Mice and Exogenous Recombinant Human SIAE Corrects Defect in Siae Mutant B Cells

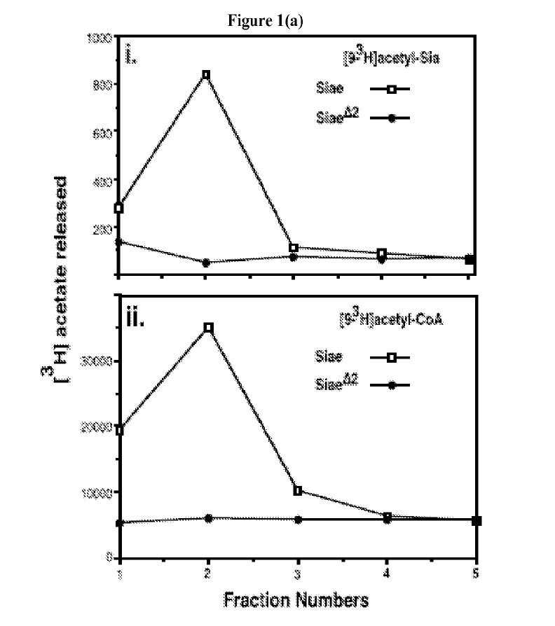

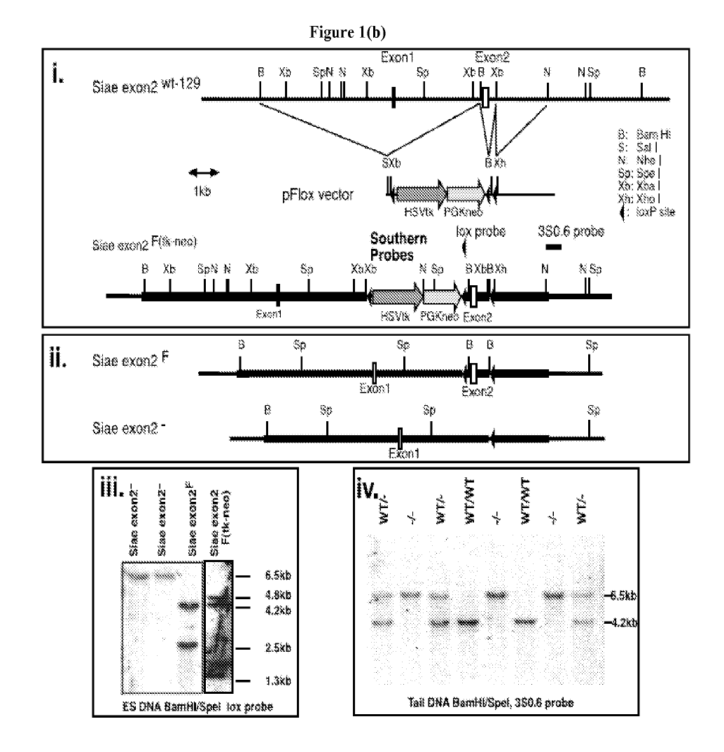

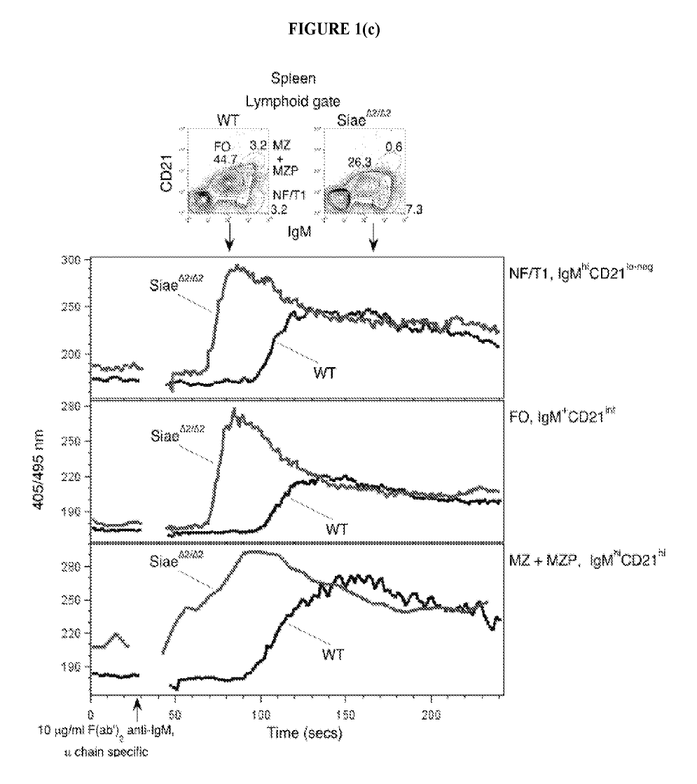

Exon 2 of the SIAE gene may be unique to the Lse splice variant. An engineered in-frame deletion of exon 2 in a murine Lse cDNA resulted in a protein that lacked esterase activity. See, FIG. 1(a). This genomic deletion of exon 2 from an Lse splice variant was achieved by homologous recombination in murine ES cells as described above. See, FIG. 1(b). Following germline transmission, homozygous null mice were generated and were found to be viable.

Since the catalytic activity of SIAE has the potential to remove 9-O-acetyl residues from α2-6-linked sialic acid containing Siglec ligands, B cells from mice lacking this esterase might exhibit enhanced B cell receptor (BCR) signaling. B cells from 5 wild type and Siae^{Δ2/Δ2} mice were gated on, and following ligation of the BCR, the accumulation of cytoplasmic calcium was analyzed using flow cytometry. As shown in FIG. 1(c), BCR cross-linking results in an accelerated and enhanced calcium flux. A similar result was seen when purified splenic B cells from mutant and WT mice were analyzed.

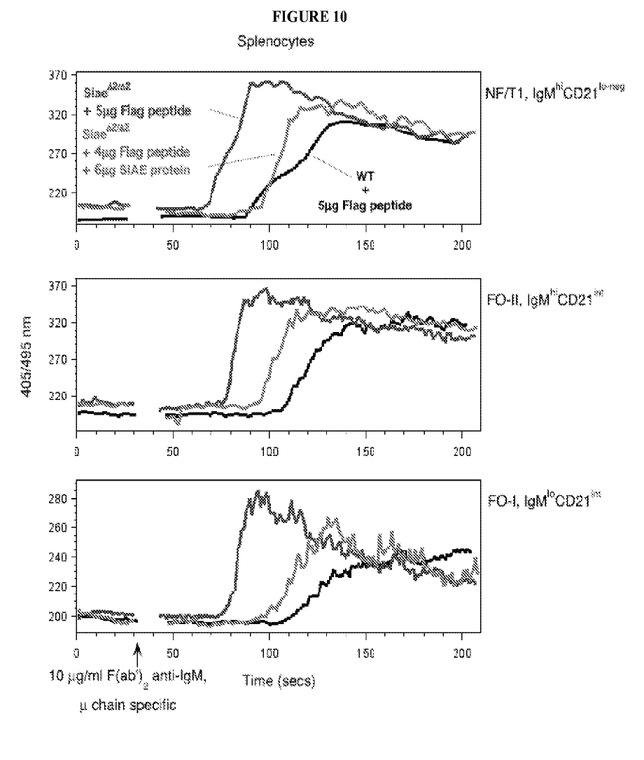

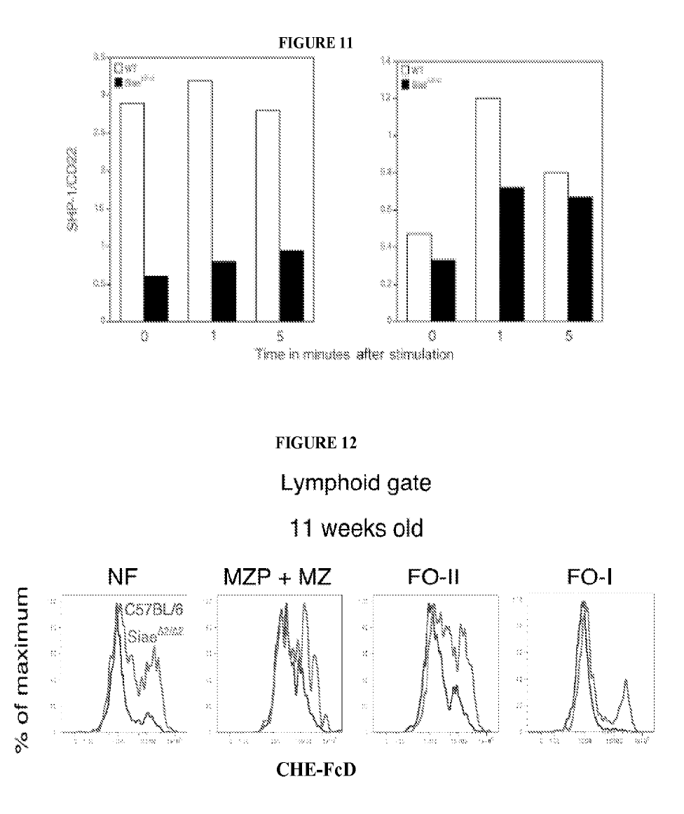

Further, the addition of SIAE protein to Siae^{Δ2/Δ2} B cells rescued the enhanced calcium influx phenomenon. Stable U2OS transfectant cell lines secreting human SIAE and affinity purified recombinant human SIAE using this source were generated (data not shown). Adding recombinant, catalytically active, SIAE to mutant murine B cells in vitro resulted in reversal of the markedly hyperactive B cell phenotype seen in B cells of the siae knockout mouse (FIG. 10). Equivalent amounts of Flag peptide were added as a control to all populations.

These data suggested that in the absence of functional Siae, BCR signal strength is markedly enhanced and that this alteration in signal strength is an intrinsic property of mutant B lymphocytes.

### Defective CD22 Signaling and Hyperacetylation of α2-6-Linked Sialic Acid Moieties on Siae Mutant B Cells

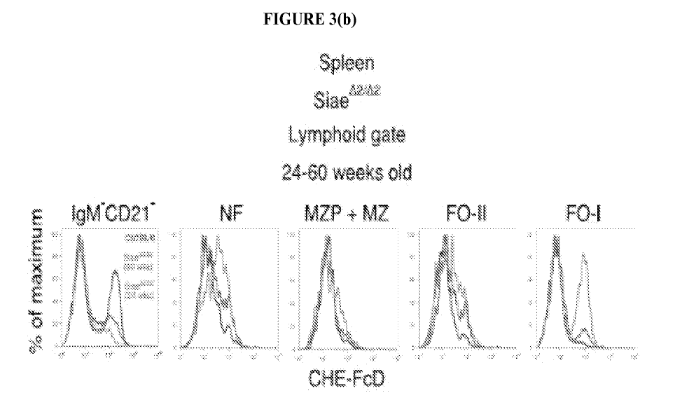

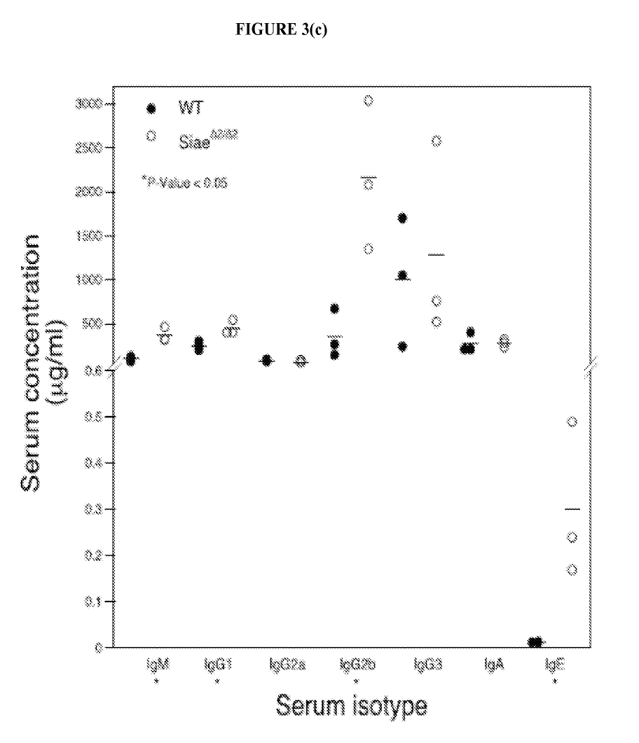

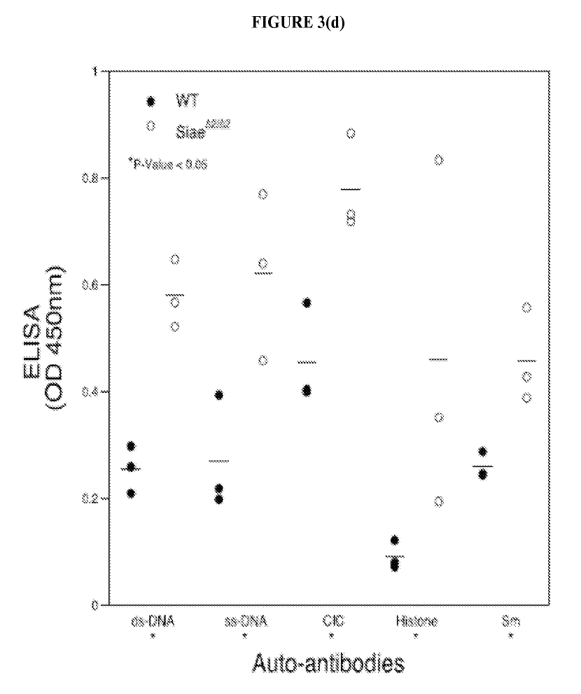

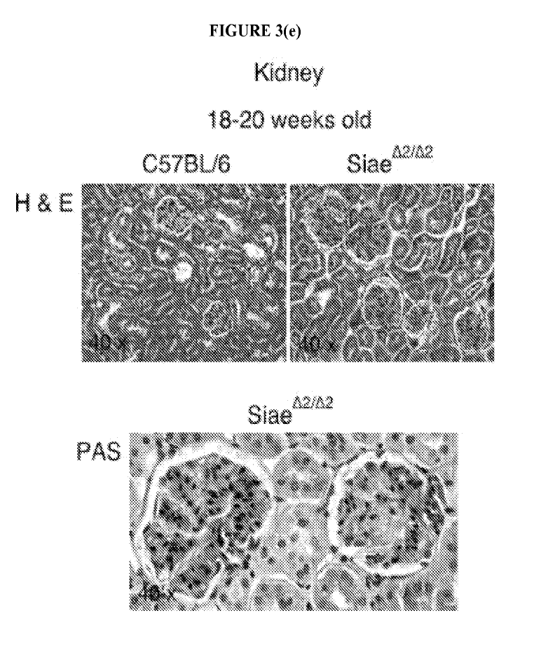

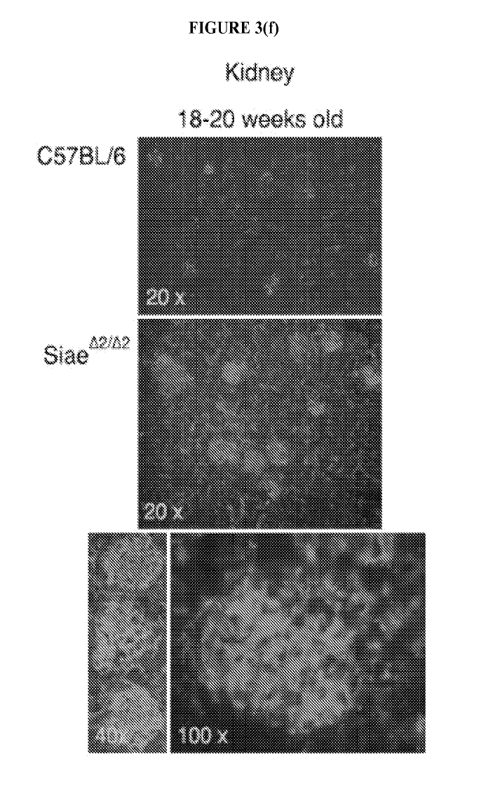

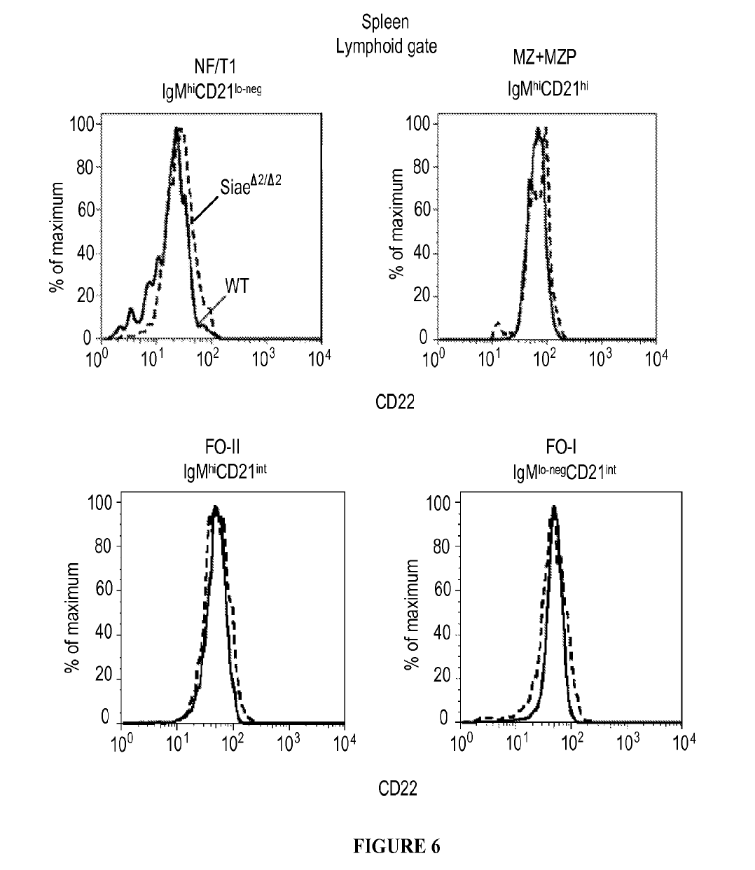

To examine defects in CD22 signaling in Siae^{Δ2/Δ2} mice, CD22 was isolated following BCR crosslinking by immunoprecipitation, and immunoprecipitates were examined for associated SHP-1 by a Western blot assay. Recruitment of SHP-1 by CD22 is markedly impaired in Siae^{Δ2/Δ2} B cells as compared with wild type B cells. As shown in FIG. 3(a), the relative amount of CD22 tyrosine phosphorylation following BCR signaling is markedly reduced in Siae^{Δ2/Δ2} mice in spite of similar levels of surface CD22 expression in WT and mutant mice (see FIG. 6). These data show that diminished SHP-1 recruitment in Siae^{Δ2/Δ2} mice is consistent with the accelerated enhancement in BCR signal strength observed in Siae^{Δ2/Δ2} B cells.

Whether a defect in Siae would result in enhanced 9-O-acetylation of α2-6 linked sialic acid in Siae^{Δ2/Δ2} mice was examined. 9-O-acetylation of sialic acid has not been previously observed on B cells, and it is likely that this modification occurs only transiently on wild type B lymphocytes. 9-O-acetylated α2-6 linked sialic acid may be detected by a diisopropyl fluorophosphate (DFP) treated fusion protein containing the Fc portion of human IgG in frame with the influenza C hemagglutinin esterase. Modification of the catalytic serine nucleophile in this fusion protein by DFP permits the viral esterase to bind to 9-O-acetyl sialic acid, but not to cleave it. This reagent, designated CHE-FcD, binds to N-linked glycans that are decorated with 9-O-acetylated α2-6-linked sialic acid moieties. The data presented herein shows detectable 9-O-acetylation of B cells in some Siae^{Δ2/Δ2} B cells, but not in wild type B cells. See, FIG. 3(b). Also as shown in FIG. 12, a consistent increase in 9-O-acetylation was detected in Siae^{Δ2/Δ2} B cells compared with WT B cells.

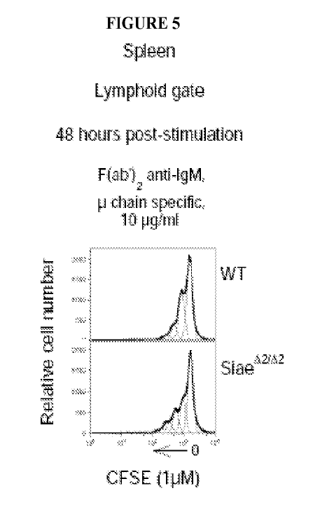

In keeping with the increased magnitude of BCR signaling, antigen receptor ligation induces enhanced proliferation of B cells in Siae^{Δ2/Δ2} mice. See, FIG. 5.

### Siae Influences Peripheral B Cell Development

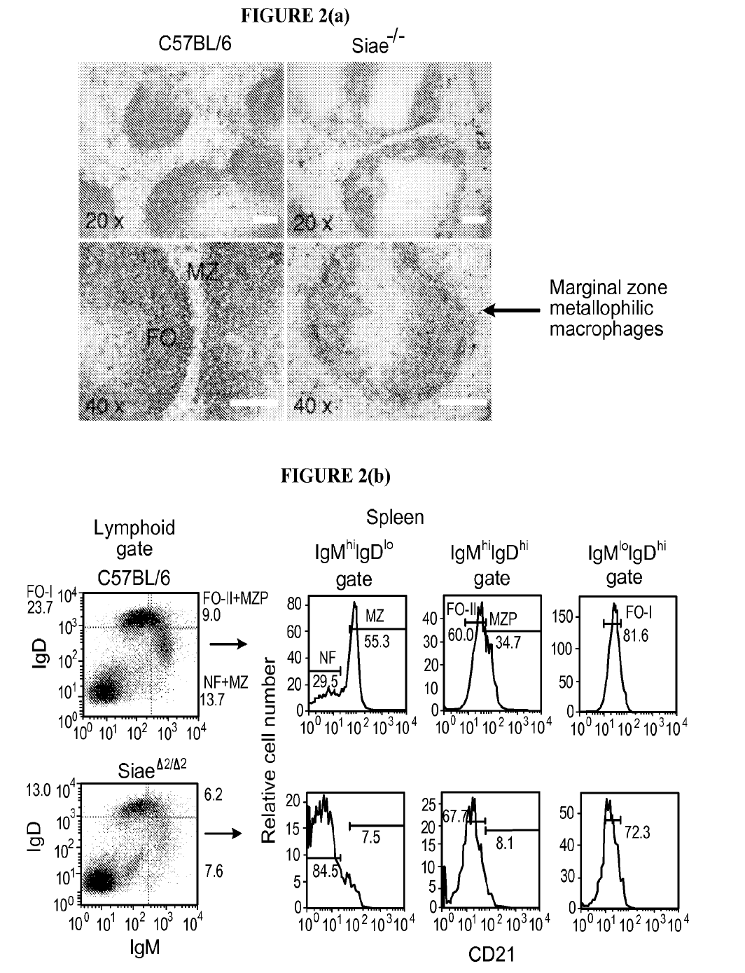

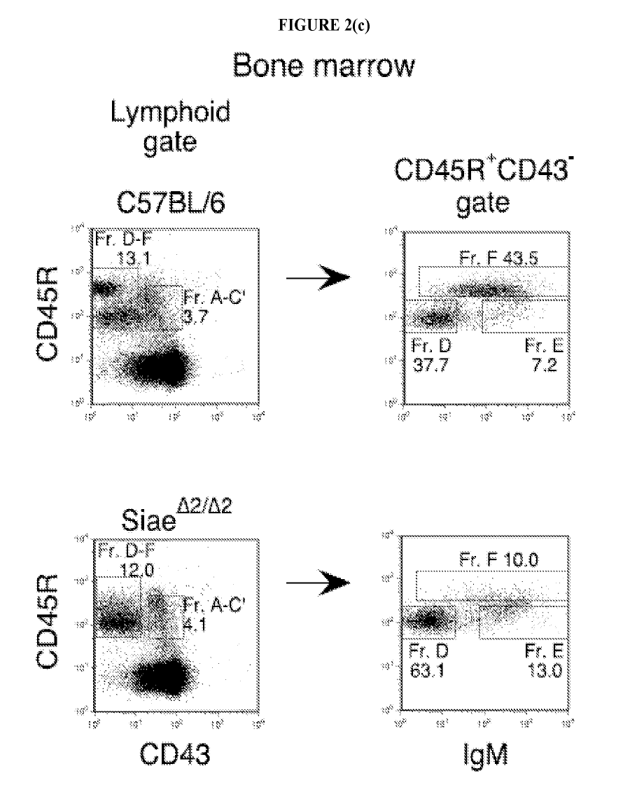

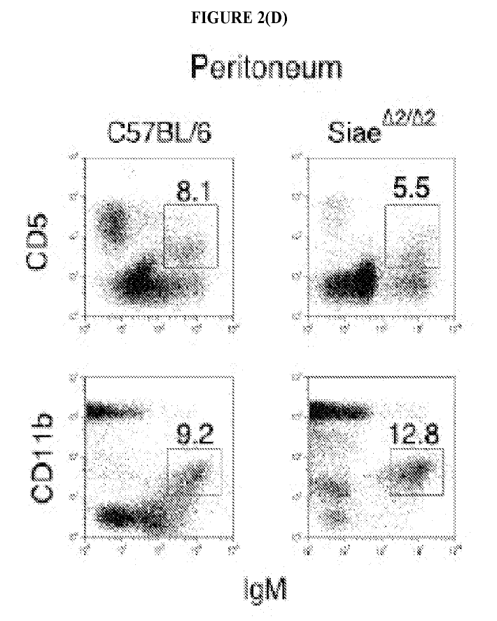

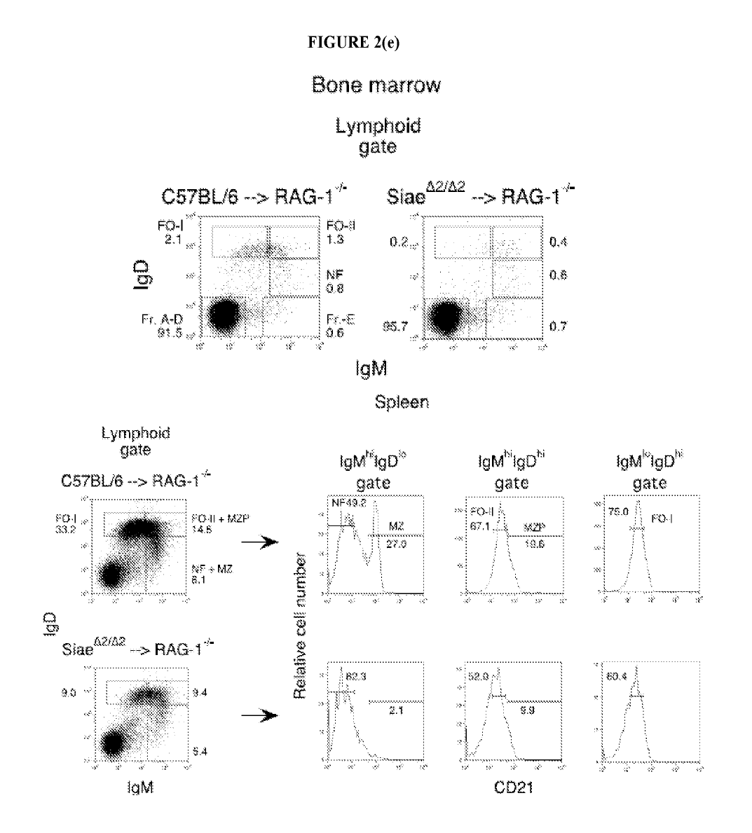

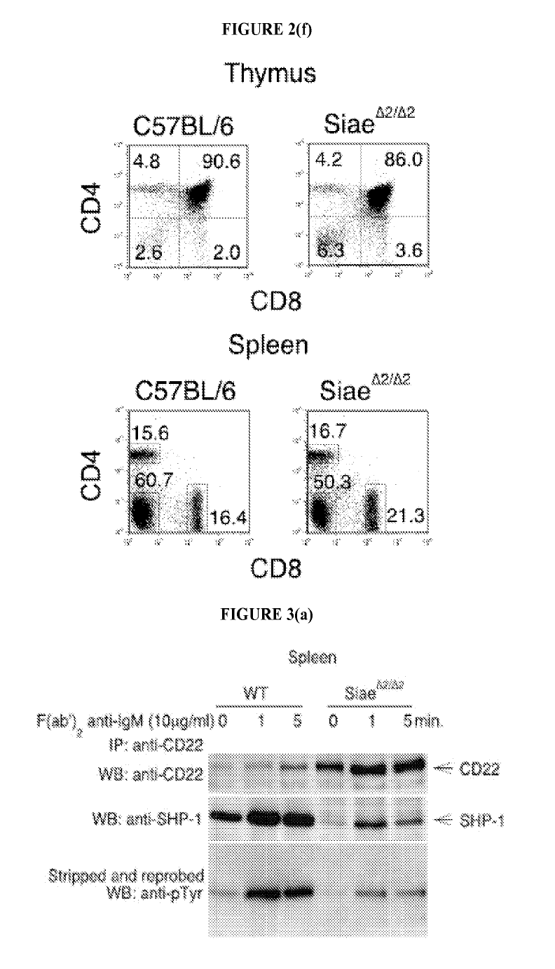

Enhanced BCR signaling in the absence of negative regulators of the BCR such as Aiolos or CD22 has been reported to lead to a loss of marginal zone (MZ) B cells (Cariappa et al., 2001. Immunity. 14: 603-615; Samardzic et al., 2002. Eur. J. Immunol. 32: 561-567). As seen in FIG. 2(a), immunohistochemistry revealed a marked reduction in splenic marginal zone B cells in Siae^{Δ2/Δ2} mice although some reduction in follicle size was also apparent. Flow cytometric analysis revealed that IgM^{hi}IgD^{hi} CD21^{hi} MZP B cells, as well as IgM^{hi}Ig^{lo}CD21^{hi} MZ B cells were markedly reduced in Siae^{Δ2/Δ2} mice. See, FIG. 2(b). An analysis of the absolute numbers of B cells in Siae^{Δ2/Δ2} mice (Table 1) revealed striking decreases in MZP and MZ B cells and more modest reductions in follicular B cells. These results are consistent with the ability of Siae to negatively regulate BCR signaling.

Follicular B cells have been reported to occupy two niches: i) the follicular niche in conventional secondary lymphoid organs; and ii) the perisinusoidal niche in the bone marrow (Cariappa et al., 2005. Immunity. 23: 397-407; Cariappa et al., 2007. Blood 109: 2339-2345). In mutant mice, including Aiolos^{-/-} mice, CD22^{-/-} mice, and CD72^{-/-} mice, all of which exhibit enhanced BCR signal strength, selective loss of recirculating follicular phenotype B cells has been seen from the perisinusoidal niche, even as these cells efficiently seed the follicular niche (Otipoby et al., 1996. Nature 384: 634-637; Sato et al., 1996. Immunity. 5: 551-562; Nitschke et al., 1997. Curr. Biol. 7: 133-143). Follicular phenotype B cell populations in the perisinusoidal niche in Siae^{Δ2/Δ2} mice were examined where a marked and selective loss of recirculating B cells in the bone marrow was observed. See, FIG. 2(c) and Table 1B. These data show a parallel phenotypic alteration previously noted in CD22 null mice where relatively strong BCR signaling is present for the development of B-1 B cells. Peritoneal B-1 B cell populations were compared between Siae^{Δ2/Δ2} and wild type mice where Siae^{Δ2/Δ2} mice showed a small increase in IgM^{hi} Mac-1⁺B-1b B cells but not of IgM^{hi}CD5⁺ B-1a B cells. See, FIG. 2(d).

TABLE 1
Absolute Numbers of B Cells in Siae^{Δ2/Δ2} Mice.

| | | |
|---|---|---|
| C57BL/6 | Siae^{Δ2/Δ2} | Decrease (fold) |

A. Absolute numbers of B cells in spleen

| | | | |
|---|---|---|---|
| Total count | 73.0 (3.8)* | 30.0 (6.3)* | 2.4 |
| Fraction | | | |
| MZ | 4.6 (0.9) | 0.2 (0.1) | 23.0 |
| NF | 2.9 (0.1) | 1.8 (0.1) | 1.6 |
| MZP | 2.1 (0.1) | 0.2 (0.1) | 10.5 |
| FO-II | 3.7 (0.2) | 1.0 (0.4) | 3.7 |
| FO-I | 15.0 (2.1) | 2.6 (0.8) | 5.8 |

B. Absolute numbers of B cells in bone marrow

| | | | |
|---|---|---|---|
| Total count | 15.4 (3.7)* | 16.9 (2.8)* | — |
| Fraction | | | |
| A-C | 7.4 (1.1) | 7.4 (3.2) | — |
| D-F | 25.5 (1.1) | 26.5 (12.7) | — |
| D | 10.6 (1.1) | 18.0 (10.6) | — |
| E | 2.1 (0.0) | 3.2 (1.1) | — |
| F | 9.5 (1.1) | 2.1 (1.1) | 4.5 |

*mean (SD), × 10⁶;
n = 3 mice per group

A lymphocyte-intrinsic defect in Siae contributes to peripheral B cell phenotypes

To determine whether the alteration in peripheral B cell populations reflected a cell-intrinsic defect in Siae function, Rag-1^{-/-} mice were reconstituted with hematopoietic stem cells from wild type and Siae^{Δ2/Δ2} bone marrow. The results of the studies showed that Siae^{Δ2/Δ2} lymphocytes in reconstituted mice exhibited the same defects in MZ B cell development and in perisinusoidal bone marrow (BM) B cells, thereby establishing that these developmental abnormalities represent lymphocyte intrinsic defects. See, FIG. 2(e). No gross defect in T cell development was discovered in the absence of Siae. See, FIG. 2(f).

### Siae Mutant Mice Exhibit Spontaneous Increases in Class-Switched Immunoglobulin and Autoantibodies

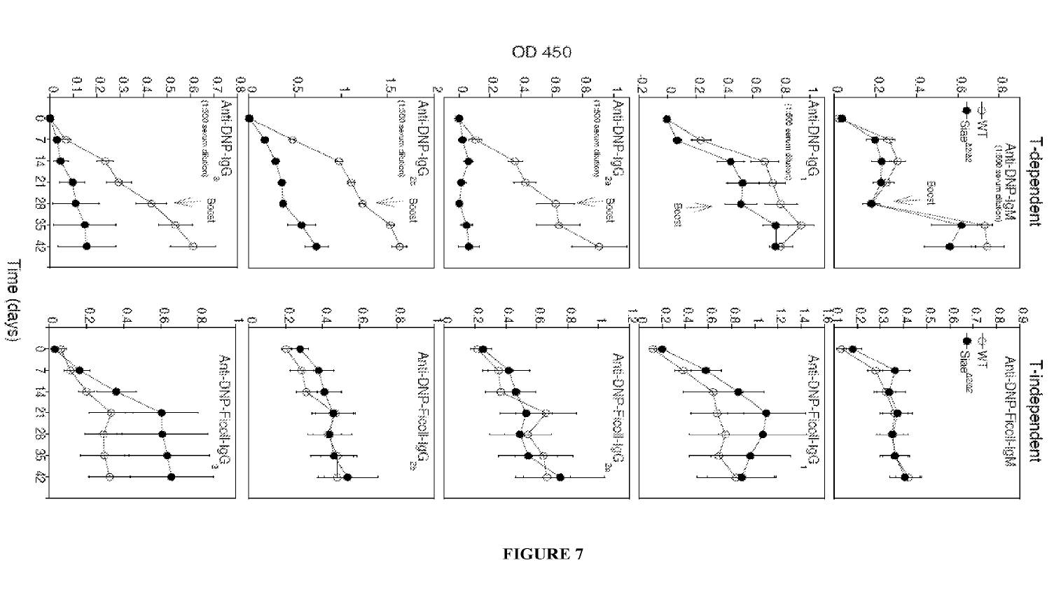

Many phenotypic features of Siae^{Δ2/Δ2} mice resemble those seen in the absence of CD22. These include, but are not limited to, enhanced BCR signaling, loss of marginal zone B cells, and/or a reduction in perisinusoidal B cells. As shown in FIG. 7, subtle alterations in responses to synthetic T dependent and T-independent antigens are observed in CD22 null mice, and a slightly altered spectrum of responsiveness to similar antigens is also noted in Siae^{Δ2/Δ2} mice. Immunization with DNP-KLH resulted in diminished IgG2a, IgG2b, and IgG3 responses in Siae mutant mice. Siae^{Δ2/Δ2} mice spontaneously developed: i) high levels of certain class-switched serum immunoglobulins such as IgE and IgG_{2b} (see FIG. 3(c)); ii) high titers of anti-nuclear antibodies and circulating immune complexes as early as at 20 weeks of age (see FIG. 3(d)); and iii) an immune complex glomerulonephritis (see FIGS. 3(e) and 3(f)).

Anti-DNA antibodies can develop in CD22 null mice after 9 months of age but glomerulonephritis is not observed using a C57B/6 genetic background (O'Keefe et al., 1999. J. Exp. Med. 189: 1307-1313). Although most of the phenotypic defects in Siae^{Δ2/Δ2} mice resemble those seen in CD22^{-/-} animals, the enhanced calcium flux after BCR activation is initiated more rapidly in the absence of this esterase. The data presented herein strongly suggest that SIAE attenuates not just CD22 function but also regulates an additional Siglec or Siglecs in B cells, and as a result Siae^{Δ2/Δ2} mice exhibit a stronger enhancement in BCR signal strength and a more severe autoimmune phenotype than that observed in CD22 null mice.

### Example 2

### Functionally Defective Germline Variants of SIAE in Autoimmunity

### Materials and Methods

### Analysis of the Sequence of SIAE

Genomic DNA was extracted from clotted blood specimens from patients with autoimmune disease using a QIAamp DNA blood mini kit (Qiagen, Valencia, Calif.) according to the manufacturer's instructions. All 10 exons of the human SIAE gene were amplified by PCR using intronic primers (Table 2). Purification of the amplified products, bidirectional automated sequencing and sequence analysis were performed as described earlier (Kwak et al., Clin Cancer Res 12, 4283-4287 (2006)). All sequence variants were confirmed by sequencing at least two independent PCR amplicons. DNAs from controls were obtained from EBV-immortalized lymphoblastoid cell-lines established from healthy blood donors (Bell, D. W. et al., Cancer Res 59, 3883-3888 (1999)).

TABLE 2
Primers for amplification and sequencing of SIAE.

| SIAE | Primer | Primer sequence | SEQ ID | Amplicon |
|---|---|---|---|---|
| exon | orientation | from 5′ to 3′ | NO: | size |
| Exon-1 | Sense | GTACAGCCCAGTCCTGAGGTCG | 6 | 339 bp |
| | Antisense | GACGCGAGCCCCTAGCCTAGCTAG | 7 | |
| Exon-2 | Sense | GACTGTATGTTCTTTGCTGTTTCAC | 8 | 344 bp |
| | Antisense | TGGATAATGTGAGCTACAGCATTAG | 9 | |
| Exon-3 | Sense | CAGAGGCTGGGAATAGCCACAAATG | 10 | 366 bp |
| | Antisense | AGAAACAGCCCTGCTTCTCCTTGTT | 11 | |
| Exon-4 | Sense | GTGGATCAAGGTGATTCAGTGCAGC | 12 | 457 bp |
| | Antisense | CCTGAAATAGTCACCATCAGGAAGG | 13 | |
| Exon-5 | Sense | GACCTCTGCCCACCCTTCTCACCTC | 14 | 380 bp |
| | Antisense | TGGGACATTCACCATATACTTAACT | 15 | |
| Exon-6 | Sense | ACAAGTGAAAGTGGATAGATGACCA | 16 | 288 bp |
| | Antisense | TGTTGCTTTAAGCCACTACGTTCTA | 17 | |
| Exon-7 | Sense | AAGACCACTACCTCAGGGCATGGAT | 18 | 342 bp |
| | Antisense | CAGGAAAGAGATCCAAATAGCACAT | 19 | |
| Exon-8 | Sense | GCAATGAGAGAAGCAGAAGCAGAGT | 20 | 359 bp |
| | Antisense | GACTCTTAAGTGCCAATCCTCAGTC | 21 | |
| Exon-9 | Sense | CTGATCACAGAGTTCAGTCAACTTT | 22 | 432 bp |
| | Antisense | CAACCAAGACCCGCCACATCGTAAT | 23 | |
| Exon-10 | Sense | CAGGTTTGTTCTGCTTACTGTAGGA | 24 | 544 bp |
| | Antisense | ATATAGAAACAGCCATGTGCTAGCT | 25 | |

### Site Directed Mutagenesis of Human SIAE and Assays for SIAE Catalytic Activity and Secretion

A human SIAE cDNA was cloned from MDA-MB 231 cells and a full length FLAG-tagged human SIAE expression construct (in pcDNA3.1) was generated. This clone was targeted for mutagenesis using PfuTurbo DNA polymerase (Stratagene, Calif., USA). Site directed mutagenesis was used to create the S127A variant with a defect in the catalytic site as well as each of the variants listed in Table 3. The PCR products were digested overnight with Dpnl (10 Units; Stratagene) and transformed into TOPIO chemically competent cells (Invitrogen, Carlsbad, Calif.). Clones containing the mutants were verified by DNA sequencing. All mutant and wild type cDNAs were transfected into HEK 293T cells. Lysates and supernatants were immunoprecipitated with anti-FLAG antibodies and catalytic activity of the immunoprecipitated esterase was assayed by a fluorimetric method (Shukla, A. K. & Schauer, R., Hoppe Seylers Z Physiol Chem 363, 255-262 (1982)). Equivalent amounts of each lysate and supernatant were immunoprecipitated for the catalytic assay as well as for quantitation of the FLAG-tagged protein by an immunoblot assay on the LI-COR Odyssey, using a mouse monoclonal anti-FLAG antibody (Sigma) and an IR Dye 800CW labeled Goat anti-mouse IgG (LI-COR) as a secondary antibody. Immunoprecipitation, metabolic labeling, and pulse chase studies were performed as described (Cariappa, A. et al. J Exp Med 206, 125-38 (2009)).

### Assays for Determining the Dominant Negative Function of Specific SIAE Variants

These assays were carried out by co-transfecting cDNAs encoding V5-tagged wild type murine Siae, together with FLAG-tagged murine versions of SIAE mutants discovered in subjects with rheumatoid arthritis into 293T cells. The V5-tagged wild type proteins in cell lysates were immunoprecipitated using mouse monoclonal anti-V5 antibody (Invitrogen), for quantitative immunoblot and esterase activity assays. Expression of FLAG-tagged mutants was also monitored by immunoprecipitation and Western blot assays.

### Statistical Analysis

Odds Ratios were determined using established methods. Two-sided p-values were calculated using Fisher's exact test.

Power Calculation:

The rate of rare mutations in diseased and control individuals are referred to as p_{D} and p_{C} respectively. Given sample sizes of n_{D} and n_{C} respectively, the power for a two-sided binomial test of the null hypothesis H₀: p_{D}=p_{C} at significance level is given by:

$$Power = Φ [ \frac{\vert {p}_{D} - {p}_{C} \vert}{\sqrt{\frac{{p}_{D} ( 1 - {p}_{D} )}{{n}_{D}} + \frac{{p}_{C} ( 1 - {p}_{C} )}{{n}_{C}}}} - {z}_{1} - \frac{α}{2} \frac{\sqrt{\overline{p} ( 1 - \overline{p} ) ( \frac{1}{{n}_{D}} + \frac{1}{{n}_{C}} )}}{\sqrt{\frac{{p}_{D} ( 1 - {p}_{D} )}{{n}_{D}} + \frac{{p}_{C} ( 1 - {p}_{C} )}{{n}_{C}}}} ]$$

(See for example Bernard Rosner's Fundamentals of Biostatistics). This power calculation is valid as long as n_{D}p_{D}≧5, n_{D}(1−p_{D})≧5, n_{C}p_{C}>5, and n_{C}(1−p_{C})≧5.

### Results

Although genome wide association studies have not revealed altered frequencies of common variants of SIAE in patients with autoimmunity, the possibility that loss-of-function variants of this gene might be enriched in patients with autoimmune disorders was addressed by complete re-sequencing of all the exons of this gene in patients with autoimmunity and in healthy controls.

Initially, 10 exons of the human SIAE gene were sequenced in 19 patients with high levels of antinuclear antibodies (including 12 patients with autoimmune disorders), referred consecutively to the Clinical Immunology Laboratory at Massachusetts General Hospital. 190 ethnically matched healthy controls were also studied.

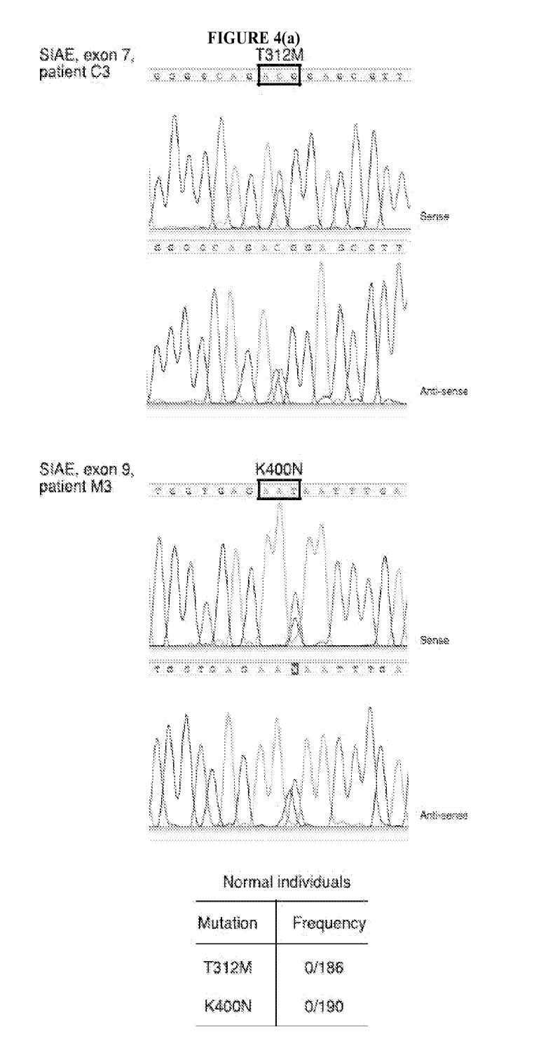

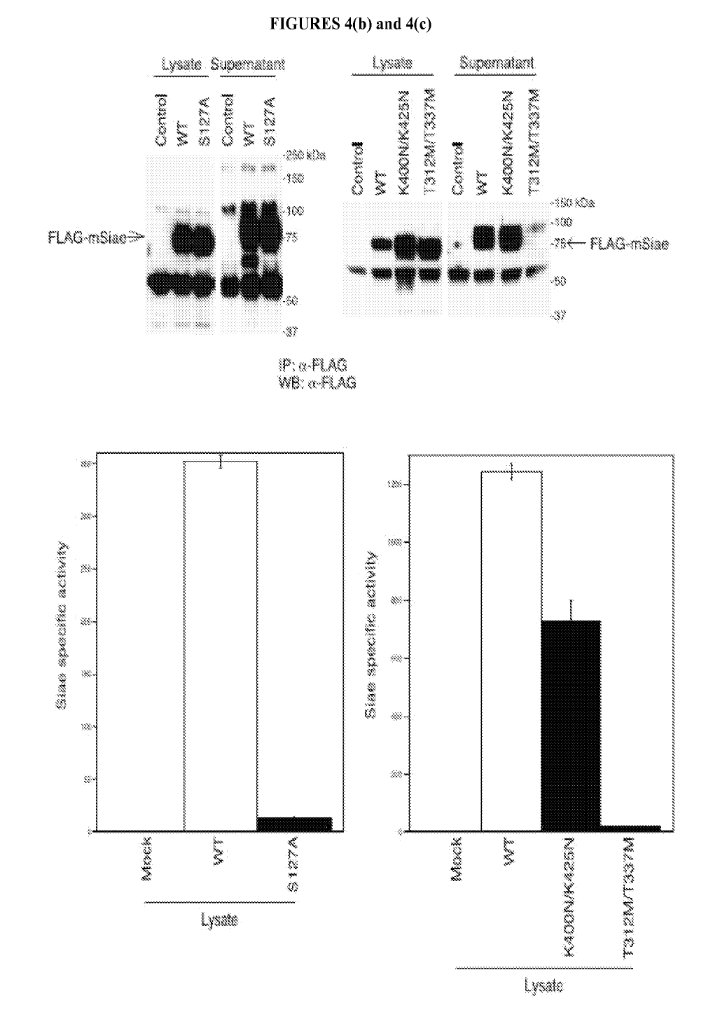

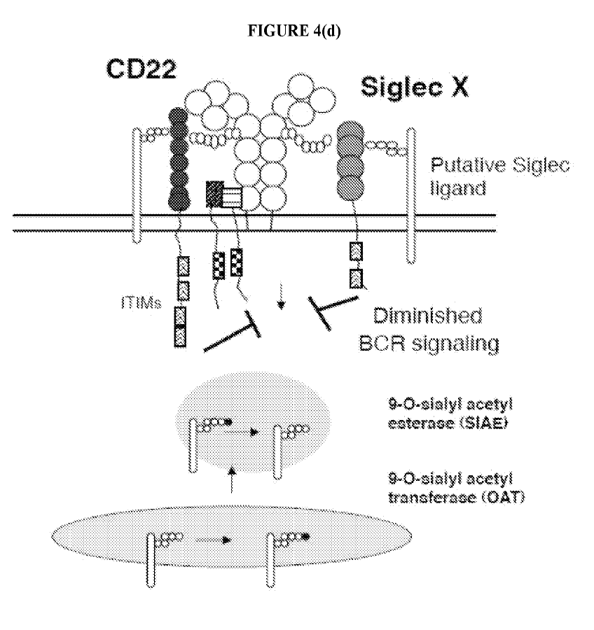

Apart from known single nucleotide polymorphisms in SIAE, approximately 6% of controls (12/190) and 1/19 patients had a conservative M89V polymorphism in SIAE. Two patients (2/19), both of whom have diagnosed autoimmune disorders (rheumatoid arthritis and thryoiditis in one individual, and Crohn's disease in the other), but none of the controls (0/190), had non-conservative changes in SIAE, a T312M substitution in one patient and a K400N alteration in the other (FIG. 4(a)).

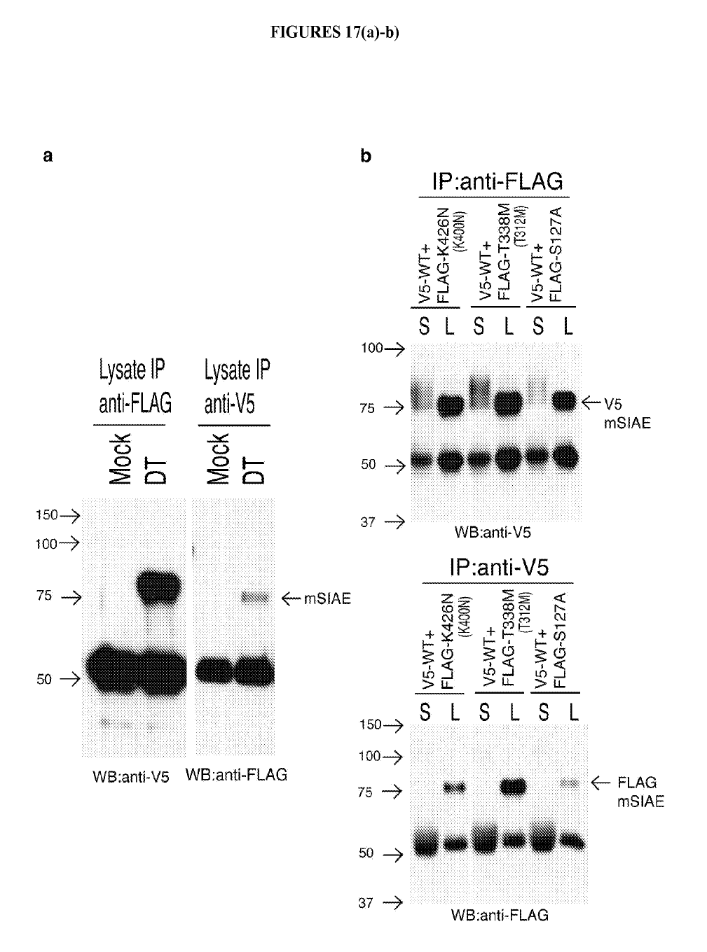

These two disease-specific substitutions were recreated in a Flag tagged murine Siae cDNA and 293T cells were transfected with cDNAs for wild type Siae, a catalytically inactive-S127A mutation 18, T312M SIAE (T337M SIAE in the mouse) and K400N SIAE (K425N SIAE in the mouse). As seen in FIGS. 4(b) and 4(c), the T312M SIAE/T337M SIAE protein is abundant in cell lysates, is relatively inefficiently secreted, and is as catalytically inactive as the active site mutation (S127A). The T312M change may be clearly considered a disease specific loss of function mutation in SIAE. The K400N change does not abrogate secretion of SIAE but this enzyme exhibits a partial defect in catalytic activity, suggesting that this alteration should also be considered a disease-linked mutation. Both mutations are heterozygous, and mutant SIAE may either potentially function in a dominant inhibitory manner or perhaps abrogate immunological tolerance by reducing functional SIAE levels below a critical threshold. Reciprocal immunoprecipitation/Western blot studies involving the use of two different epitope tagged versions of SIAE indicate that SIAE exists as a dimer or a higher order oligomer (see FIG. 17), supporting the notion that mutant forms of SIAE may have the potential to function in a dominant inhibitory manner.

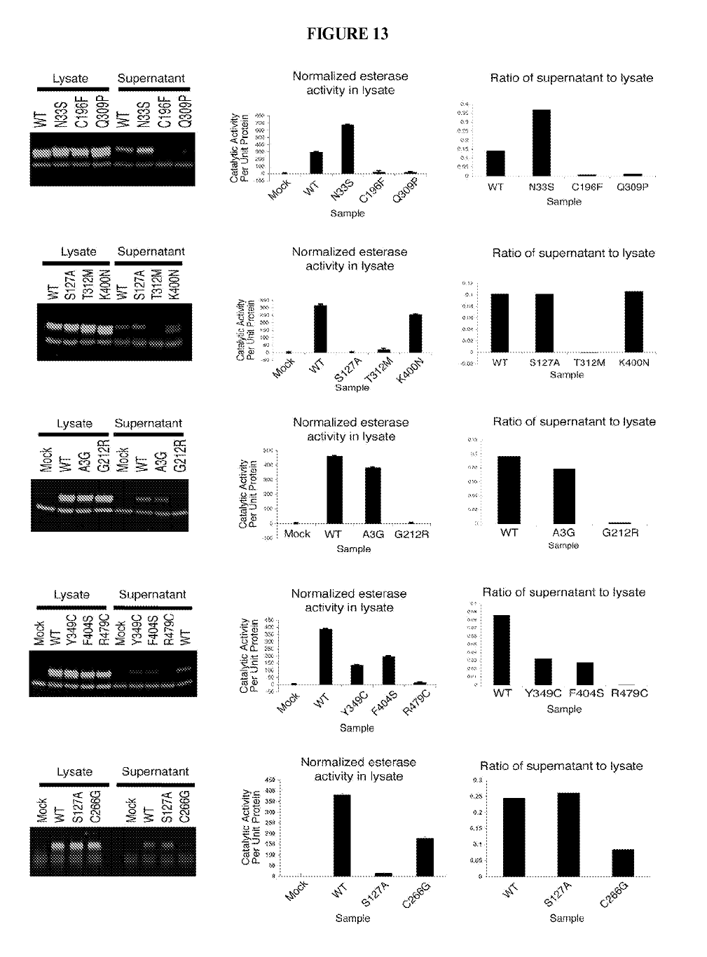

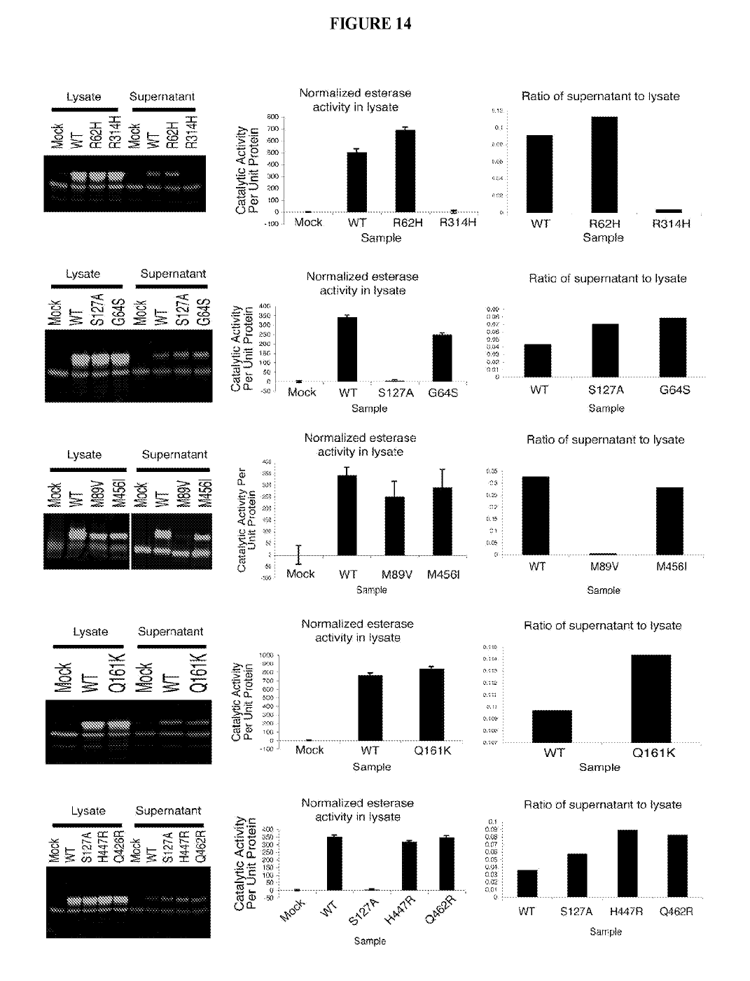

76 Caucasian subjects with rheumatoid arthritis from the NARAC (North American Rheumatoid Arthritis Consortium) collection, and 89 subjects with inflammatory bowel disease (IBD) from MGH were next analyzed, making a total of 188 autoimmune subjects. The 190 control DNAs in this study were obtained from healthy volunteers at MGH primarily of European ancestry. Re-sequencing of all 10 exons of SIAE revealed point substitutions in SIAE in both patients and controls, and the function of variant enzymes with each of these substitutions was assessed (described below, see FIGS. 13 and 14, and Table 3).

Since the initial analyses revealed a marked enrichment of loss of function SIAE variants in autoimmune subjects as compared with controls, power calculations were undertaken to determine what would be an optimal sample size, and then the analysis of a larger number of patients and controls was undertaken. Autoimmune subjects in this larger study included patients with rheumatoid arthritis from NARAC, subjects from the MADGC (Multiple Autoimmune Disease Genetics Consortium) collection with systemic lupus erythematosus (SLE) and juvenile idiopathic arthritis (JIA), and subjects from MGH with SLE, or RA or IBD. The study also included subjects with multiple sclerosis (MS) from a collection at the Brigham and Womens' Hospital, and subjects with Type 1 diabetes from the EDIC (Epidemiology of Diabetes Intervention and Complication study) collection of the NIDDK (National Institute of Diabetes, Digestive and Kidney Diseases). Additional healthy control DNAs, primarily from subjects of European ancestry, were obtained from the MGH Cancer Center, from the Feinstein Institute, and from the phenogenetic collection at Brigham and Womens' Hospital.

In order to determine whether variants were functional or defective, C-terminal Flag-tagged human SIAE cDNAs cloned from MDA-MB 231 cells containing the changes corresponding to the coding SIAE variants that were discovered in patients and controls were created. Each cDNA was transfected into 293T cells, and lysate and supernatants were each divided into two equal aliquots. SIAE was immunoprecipitated with anti-Flag antibodies, and one aliquot was saved for a quantitative Western blot assay while the other was utilized in an esterase assay using a fluorogenic substrate, 4-methylumbelliferyl acetate. Quantitative Western blotting was performed using a near infrared-dye labeled second antibody and detected using the Li-Cor Odyssey system. Each cDNA was transfected three or more times and the entire assay performed on at least three occasions for each cDNA.

As described in Table 3, 24/820 autoimmune subjects were identified with non-synonymous substitutions in SIAE that did not represent known SNPs. In 21 of these patients, the SIAE variants were found to be functionally defective either because of a defect in catalytic activity to below 50% of wild type or because of a profound defect in secretion (in the absence of a catalytic defect). A group of missense variants that are severely catalytically defective include c.935C>T, c.587G>T, c.926A>C, c.634G>A and c.1435C>T and encode T312M SIAE, C196F SIAE, Q309P SIAE, G212R SIAE, and R479CSIAE, respectively. The analysis of these severely catalytically defective variants by transfection, immunoprecipitation, enzyme assays and immunoblot assays are shown in FIG. 13. These variants are also very poorly secreted presumably because they are grossly misfolded proteins that fail to egress the endoplasmic reticulum.

More modest, but reproducible catalytic defects were seen in the c.1046A>G variant that encodes Y349C SIAE, and this variant also exhibits reduced secretion (see FIG. 13, bottom panels). The c.1211T>C variant encodes F404S SIAE that also appears to exhibit a less severe catalytic defect (but nevertheless below the 50% cutoff set), similar to that seen in Y349C SIAE (FIG. 13). F404S SIAE was found in four autoimmune patients including two blood relatives, one with SLE and the other with juvenile idiopathic arthritis. The c796T>G variant found in one subject with Type I diabetes encodes C266G SIAE which is also defective (FIG. 13).

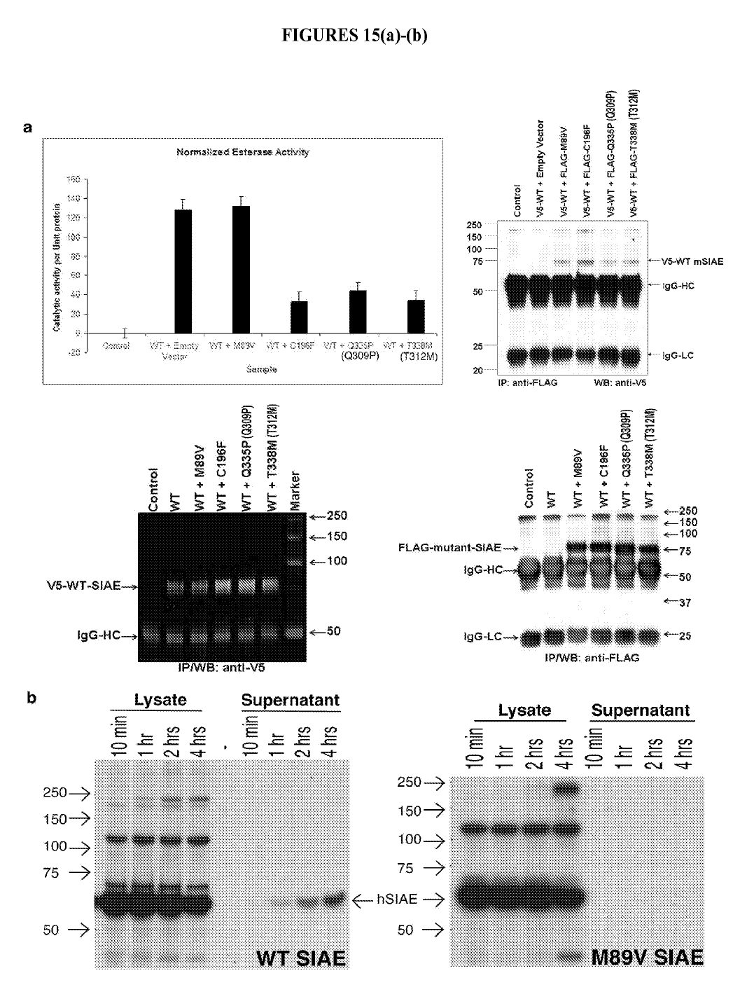

In contrast to the catalytically defective SIAE variants seen in patients with autoimmune diseases, with the exception of two variants (R314H and T312M) observed once each in controls (Table 3), most of the new SIAE variants found in normal subjects did not exhibit reduced catalytic activity, as shown in FIG. 14. Interestingly, one of these variants, M89V SIAE, is catalytically active but is not secreted (see FIG. 14, third set of panels). The M89V variant is quite common in controls in the heterozygous state (9.7%, see Table 4). In order to more precisely establish that M89V SIAE is secretion defective, 293T cells transfected with wild type and M89V SIAE respectively were metabolically labeled with ³⁵[S]methionine and chased for 10 min, 1 h, 2 h and 4 h. As seen in FIG. 15(b), a striking defect in secretion of M89V SIAE was confirmed by this analysis.

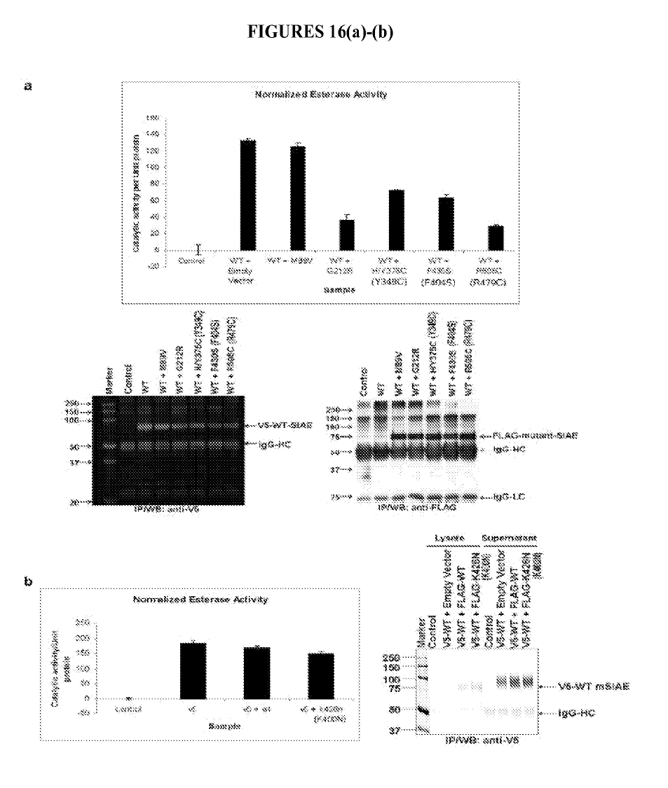

Since SIAE exists as a dimer or higher order oligomer (FIG. 17), we examined whether catalytically dead mutants from patients with autoimmunity on the one hand, and the catalytically active but secretion-defective M89V variant on the other, could function in a dominant interfering manner (FIG. 15(a)). The K400N allele (FIG. 13) was also tested as a representative catalytically normal SIAE allele. Since the ultimate test of dominant negative function would be to re-create a heterozygous animal with one mutant allele, mutations were recreated in a murine Siae cDNA for these studies. As seen in FIG. 15(a) and FIG. 16, the murine equivalents of the C196F, G212R, Q309P, T312M, Y349C, F404S and R479C variants are capable of dominantly inhibiting wild type SIAE, while M89V SIAE and K400N SIAE are not. Based on this finding, it was clear that only subjects with homozygous M89V/M89V SIAE variants (as opposed to subjects with heterozygous M89V changes) should be considered to be of potential functional relevance for predisposition to autoimmunity. The cysteine at position 266 in humans is not conserved in rodents, and the C266G variant is the only dysfunctional disease related point substitution that was not tested for dominant interfering activity.

Strikingly, eight patients (3 with RA, 1 with SLE, 1 with MS, and three with Type 1 diabetes) are homozygous for c.[265A>G]+[265A>G] alleles, which encode M89V/M89V SIAE variants, whereas these homozygous genotypes were not observed in a single control. Given the defect in secretion of this variant, in subjects with homozygous M89V/M89V SIAE, it is likely that this esterase is unlikely to be able to effectively access the post-Golgi compartment in which it would normally de-acetylate 9-O-acetylated sialoproteins that serve as CD22 ligands.

A number of SIAE variants were found in patients with autoimmunity that are probably not involved in the genetic predisposition of these subjects to autoimmunity. For example, a c.98A>G variant encoding N33S SIAE was discovered in a patient with RA and was found to be functionally normal based on the criteria used (FIG. 13, top panels). One patient with Crohn's disease inherited a c.8C>G variant encoding an A3G change in the signal peptide portion of SIAE. The coding region of SIAE would be predicted to be intact in this variant though it is theoretically possible that A3G SIAE might not be readily translocated into the ER. It is unlikely that A3G SIAE is translocation-defective given the accumulation of SIAE in culture supernatants when A3G SIAE is transfected into 293 T cells (FIG. 13, third set of panels). A c.1200G>T variant that encodes K400N SIAE was discovered in a patient with Crohn's disease initially examined as part of a small subset of patients with high ANA titers. This enzyme is active and is efficiently secreted but always appears in supernatants as a protein doublet (FIG. 13, second set of panels). Lysine 400 is immediately adjacent to a consensus N-glycosylation site, and it may be that a particular N-glycan is added inefficiently in this variant. This variant is however catalytically active and we classify it as a non-defective allele.

In the first phase of this study defective variants were identified in 7/188 autoimmune patients and 0/190 controls. The Odds Ratio could only be calculated as an estimate (Peto Odds Ratio), and this approach yielded an Odds Ratio of 7.71. In the second phase of the study 14/632 autoimmune patients and 2/458 controls inherited defective SIAE alleles, and the calculated Odds Ratio was 5.17. The total number of patients with autoimmune disorders analyzed was 820, with 21/820 inheriting defective SIAE alleles, and the total number of ethnically matched controls was 648, with 2/648 controls inheriting defective SIAE alleles. The calculated Odds Ratio for all autoimmune disorders was 8.49 with a two-sided p-value of 0.0004.

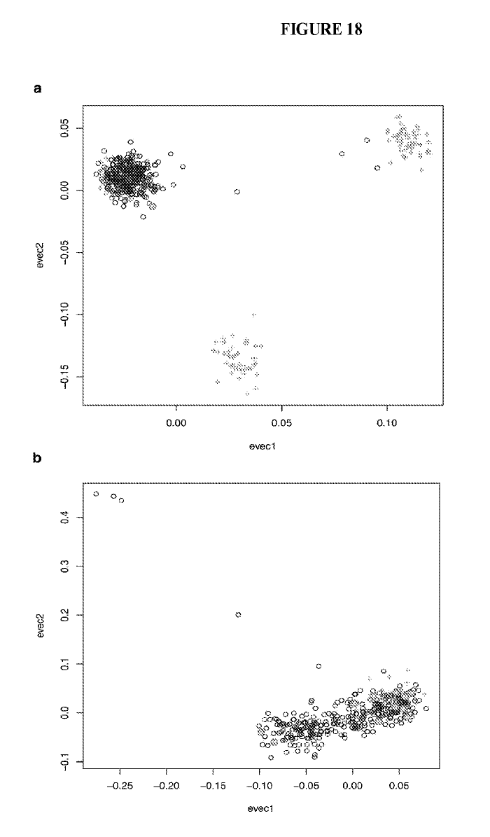

While care was taken to include only Caucasian non-Jewish subjects in this study, an objective determination of shared ethnicity, a principal components analysis, was conducted on samples with defective SIAE alleles and on controls. An analysis was run with markers informative for major ancestry and this showed three separate groups (crosses) for HAP map samples (FIG. 18). The Caucasian cluster in the upper left portion of FIG. 18(a) clusters with almost all controls (black circles) and all cases with defective SIAE (grey circles); the African cluster is in the upper right portion, and the Asian cluster is below. A second analysis using only Caucasian clustered samples (FIG. 18(b)) showed that the cases and controls cluster similarly using markers that capture European diversity; most of this diversity relates to north-south ancestry differences, giving the spread along the x-axis in the plot. There were a few outliers in the controls, but none in the positive cases with SIAE mutations. Thus, the novel SIAE variants that were observed in subjects with autoimmunity cannot be ascribed to population stratification with respect to controls.

Ten of the 648 controls inherited a non-synonymous rare variant of SIAE but only 2/648 inherited defective alleles (FIG. 14 and Table 3). One of the rare variants (c.935C>T encoding T312M SIAE) found in one of the controls (all 10 exons were sequenced in 648 controls) was identical to a defective variant originally found in a patient with RA and also in a patient with MS. Another variant, (c.941G>A encoding R314H SIAE), was found in a single control and was also found to be defective (FIG. 14). The remaining rare variants found in controls (c.1340A>G, c.90G>A, c.481C>A, c.185G>A, c.1368G>A, and c.1385A>G encoding the H447R, G64S, Q161K, R62H, M4561 and Q462R versions of SIAE respectively) were completely normal as determined by the two assays described (see FIG. 14 and Table 3 for a summary).

All the defective heterozygous SIAE alleles in patients that were tested function in a dominant interfering manner in the transfection assay employed. While high Odds Ratios were observed in a number of disorders, the results from rheumatoid arthritis subjects (Odds Ratio, 8.3, 2-tailed p=0.0056) and Type I diabetes patients (Odds Ratio, 9.3, 2-tailed p=0.0064) were particularly significant. While a dominant negative effect may contribute to disease susceptibility for all tested defective heterozygous variants, haploinsufficiency might be a possible mechanism for some of the defective variants which might result in the reduction of levels of catalytically active enzyme in B cells below a tipping point. This would imply that the W48X alteration found in a patient with type I diabetes (Table 3) may be clinically relevant.

A number of susceptibility loci for human autoimmune disorders have been uncovered by genome wide association studies, and the relative risks for these associations are generally modest (Cohen, J. C. et al., Science 305: 869-72 (2004); Gregersen, P. K. & Behrens, T. W., Nat Rev Genet. 7: 917-28 (2006)). A number of recent reports have supported the hypothesis that rare genetic variants can contribute to disease susceptibility. A pioneering study on rare variants in genes that are relevant to lipoprotein synthesis in patients susceptible to cardiovascular disease utilized a predictive algorithm (Polyphen) to determine which variants were probably non-functional (Altshuler et al., Science 322: 881-8 (2008)). Loss-of-function variants in the Trex gene, which has a single coding exon, have also been described in patients with SLE and a recent re-sequencing study has revealed rare variants in the cytosolic helicase MDA5/1F1H1, which mediates innate immune responses to pathogen encoded RNAs. The data described herein provide important support for a role for rare variation in the predisposition to autoimmune diseases and strikingly illustrate the importance of performing functional assays for the variants being studied. All of the variants identified through re-sequencing are listed in Tables 4 and 5. There is clear enrichment of defective coding variants in autoimmune patients as compared to controls (Table 6), and it is notable that these variants primarily involved residues that are highly conserved across evolution. By virtue of the larger sample sizes, rheumatoid arthritis and Type I diabetes show the most compelling evidence for association, but nominal evidence of association is also present for several other specific disorders (Table 7).

The contribution of B cells to disease, with or without a role for auto-antibodies, is recognized in a growing number of diseases including rheumatoid arthritis, multiple sclerosis, and Type I Diabetes. As shown above, mutant Siae in rodents results in enhanced B cell activation and a break in B cell tolerance, but it remains formally possible that SIAE may be required in cell types other than B cells in humans as well as in rodents. One type of inflammatory bowel disease, Crohn's disease, like multiple sclerosis, is generally considered to be etiologically linked to T_{H}1 or T_{H}17 cells (Xavier et al., Nature 448: 427-34 (2007)). Although B cell depletion can result in marked clinical improvement in patients with multiple sclerosis (Hauser, S. L. et al., N Engl J Med 358: 676-88 (2008)). B cells are not generally considered to be of etiopathogenic significance in Crohn's disease; it remains formally possible that autoantigen specific B cells may function as critical antigen presenting cells that secrete cytokines driving helper T cell polarization in certain disease situations.

### Example 3

### Level of 9-O-acetylated B Cell in Patients with Autoimmune Disorders

Flow cytometry based method using CHE-Fc-D as described above can be used to quantitate 9-O-acetyl sialic acid on human B cells (see FIG. 20). The method was used to quantitate 9-O-acetyl sialic acid on human B cells from normal subjects and SLE (systemic lupus erythematosus) patients. As shown in FIG. 21, some of the SLE patients tested exhibited increased 9-O-acetylation of B cells. It is expected that 9-O-acetylation of B cells would be increased in some patients with a defect in the synthesis or function of SIAE (because of, e.g., mutations such as those described above).

### Example 4

### Quantifying Serum Level of SIAE in Human Patients

As demonstrated above, SIAE is a secreted protein and SIAE alleles encode polypeptides with secretion defects were identified in autoimmune patients. Western blot assays using a specific polyclonal antibody (murine) against human SIAE were used to detect human serum SIAE (see FIG. 22). As seen in FIG. 22, Flag-tagged SIAE, as expected, runs a little slower than SIAE in human serum. These polyclonal antibodies have been tested on Western blot assays and shown to be highly specific for SIAE (data not shown). This antiserum also recognizes human SIAE made in E. coli, suggesting that the reagent recognizes non-glycosyl protein epitopes in SIAE. The LICOR-Odyssey system was used to facilitate quantitation—the anti-mouse IgG second antibody is conjugated to IR-CMW 800, an infrared fluorescent dye that allows quantitation over a wide linear range (data not shown).

These data demonstrate that SIAE can be readily detected in the serum of human subjects.

TABLE 3
SIAE Variants in Caucasian Autoimmune Patients and Controls.

| | | | | | |
|---|---|---|---|---|---|
| | Esterase | | Dom. | | |
| SIAE change | activity | Secretion | Neg. | Disease | Source |

Autoimmune Patients (n = 820)

| | | | | | |
|---|---|---|---|---|---|
| T312M | Defective | Defective | Yes | RA | MGH |
| T312M | Defective | Defective | Yes | MS | BWH |
| Q309P | Defective | Defective | Yes | RA | NARAC |
| C196F | Defective | Defective | Yes | RA | NARAC |
| M89V/M89V | Normal | Defective | No | RA | NARAC |
| M89V/M89V | Normal | Defective | No | RA | NARAC |
| M89V/M89V | Normal | Defective | No | RA | NARAC |
| M89V/M89V | Normal | Defective | No | SLE | MADGC |
| M89V/M89V | Normal | Defective | No | MS | BWH |
| M89V/M89V | Normal | Defective | No | T1D | NIH |
| M89V/M89V | Normal | Defective | No | T1D | NIH |
| M89V/M89V | Normal | Defective | No | T1D | NIH |
| G212R | Defective | Defective | Yes | CD | MGH |
| F404S | Defective | Defective | Yes | JIA | MADGC |
| F404S | Defective | Defective | Yes | SLE | MADGC |
| F404S | Defective | Defective | Yes | UC | MGH |
| F404S | Defective | Defective | Yes | MS | BWH |
| Y349C | Defective | Reduced | Yes | SLE | MADGC |
| R479C | Defective | Defective | Yes | CD | MGH |
| W48X | Truncated/ | Truncated/NT | NT | T1D | NIH |
| | NT | | | | |
| C266G | Defective | Defective | NT | T1D | NIH |
| K400N | Normal | Doublet | No | CD | MGH |
| A3G | Normal | Normal | NT | CD | MGH |
| N33S | Normal | Normal | NT | RA | NARAC |

Ethnically Matched Controls (n = 648)

| | | | | | |
|---|---|---|---|---|---|
| R314H | Defective | Defective | NT | Control | NS/LIJ |
| T312M | Defective | Defective | Yes | Control | NS/LIJ |
| Q161K | Normal | Normal | NT | Control | MGH |
| G64S | Normal | Normal | NT | Control | MGH |
| G64S | Normal | Normal | NT | Control | MGH |
| G64S | Normal | Normal | NT | Control | MGH |
| G64S | Normal | Normal | NT | Control | MGH |
| G64S | Normal | Normal | NT | Control | NS/LIJ |
| Q462R | Normal | Normal | NT | Control | MGH |
| H447R | Normal | Normal | NT | Control | MGH |
| R62H | Normal | Normal | NT | Control | NS/LIJ |
| M456I | Normal | Normal | NT | Control | NS/LIJ |

TABLE 4
SIAE variant frequencies in Controls (n = 648)

| | Amino Acid | | |
|---|---|---|---|
| Sequence Variant | Substitution | # in Controls | % of pop |
| 5′UTR − 36T > G | — | 1 | 0.154 |
| 5′UTR − 11T > A | — | 1 | 0.154 |
| IVS1 + 83C > T | — | 1 | 0.154 |
| IVS1 − 31T > C | — | 1 | 0.154 |
| c.185G > A | G62R | 1 | 0.154 |
| c.190G > A | G64S | 9 | 1.389 |
| c.212A > G | K71R | 21 | 3.241 |
| IVS2 + 19C > T | — | 9 | 1.389 |
| IVS2 + 20G > A | — | 255 | 39.352 |
| IVS2[+20G > A] + [+20G > A] | — | 31 | 4.784 |
| IVS2 + 45C > T | — | 13 | 2.006 |
| c.265A > G | M89V | 63 | 9.722 |
| c.327C > T | T109T | 1 | 0.154 |
| IVS3 − 102G > A | — | 2 | 0.309 |
| c.467T > C | S156S | 58 | 8.951 |
| c.481C > A | Q161K | 1 | 0.154 |
| IVS4 + 18C > T | — | 3 | 0.463 |
| IVS4 + 20T > C | — | 1 | 0.154 |
| c.573C > T | Y191Y | 5 | 0.772 |
| c.633C > T | I211I | 2 | 0.309 |
| IVS5 + 37C > T | — | 27 | 4.167 |
| IVS5 + 65C > T | — | 16 | 2.469 |
| IVS5 − 23C > G | — | 1 | 0.154 |
| c.691C > T | I297I | 1 | 0.154 |
| c.935C > T | T312M | 1 | 0.154 |
| c.941G > A | R314H | 1 | 0.154 |
| IVS7 + 39C > T | — | 11 | 1.698 |
| IVS7 + 103C > G | — | 1 | 0.154 |
| c.1020T > C | R340R | 6 | 0.926 |
| IVS8 + 81A > G | 81+ | 37 | 5.710 |
| IVS8 − 68C > T | — | 15 | 2.315 |
| IVS8 − 66T > C | — | 8 | 1.235 |
| IVS9 − 23C > T | — | 26 | 4.012 |
| c.1340A > G | H447R | 1 | 0.154 |
| c.1368G > A | M456I | 1 | 0.154 |
| c.1390G > C | L464L | 1 | 0.154 |
| c.1385A > G | Q462R | 1 | 0.154 |
| c.1400C > T | A467V | 6 | 0.926 |
| c.1452A > G | T484T | 53 | 8.179 |
| c.1476C > T | C492C | 1 | 0.154 |
| IVS10 * 61G > C | — | 1 | 0.154 |
| IVS10 * 118G > A | — | 1 | 0.154 |

TABLE 5
SIAE Variant Frequencies in Autoimmune Subjects (n = 820)

| | Amino Acid | | |
|---|---|---|---|
| Sequence Variant | Substitution | # in Patients | % of pop |
| IVS0 − 11T > A | — | 1 | 0.122 |
| IVS1 + 71C > T | — | 1 | 0.122 |
| c.8C > G | A3G | 1 | 0.122 |
| IVS1 − 28C > A | 28+ | 5 | 0.610 |
| c.98A > G | N33S | 1 | 0.122 |
| c.143G > A | W48X | 1 | 0.122 |
| c.212A > G | K71R | 4 | 0.489 |
| c.[212A > G] + [212A > G] | K71R | 1 | 0.122 |
| IVS2 + 20G > A | — | 303 | 36.951 |
| IVS2[+20G > A] + [+20G > A] | — | 35 | 4.268 |
| IVS2 + 45C > T | — | 9 | 1.098 |
| c.265A > G | M89V | 61 | 7.439 |
| c.[265A > G] + [265A > G] | M89V | 8 | 0.976 |
| c.467T > C | S156S | 49 | 5.976 |
| IVS4 + 10G > A | — | 2 | 0.244 |
| IVS4 + 18C > T | — | 3 | 0.366 |
| c573C > T | Y191Y | 4 | 0.488 |
| c.587G > T | C196F | 1 | 0.122 |
| c.634G > A | G212R | 1 | 0.122 |
| IVS5 + 37C > CT | — | 36 | 4.390 |
| IVS5 + 65C > CT | — | 23 | 2.805 |
| IVS5 − 23C > G | — | 1 | 0.122 |
| c.796T > G | C266G | 1 | 0.122 |
| c.926A > C | Q309P | 1 | 0.122 |
| c.935C > T | T312M | 2 | 0.244 |
| IVS7 + 39C > T | — | 9 | 1.098 |
| c.1020T > C | R340R | 3 | 0.366 |
| c.1041C > T | F347F | 3 | 0.366 |
| c.1046A > G | Y349C | 1 | 0.122 |
| IVS8 + 81A > G | — | 26 | 3.171 |
| IVS8 − 68C > T | — | 15 | 1.829 |
| IVS8 − 66T > C | — | 19 | 2.317 |
| IVS8 + 181A > G | — | 5 | 0.610 |
| c1200G > T | K400N | 1 | 0.122 |
| c.1211T > C | F404S | 4 | 0.488 |
| IVS9 − 23C > T | — | 49 | 5.976 |
| c.1400C > T | A467V | 2 | 0.247 |
| c.1404C > A | I468I | 1 | 0.122 |
| c.1435C > T | R479C | 1 | 0.122 |
| c.1452A > G | T484T | 49 | 5.976 |
| c.1482A > G | L494L | 1 | 0.122 |
| IVS10 * 62C > G | — | 1 | 0.122 |
| IVS10 * 118G > A | — | 4 | 0.489 |

TABLE 6
Functionally defective SIAE coding variants in rheumatoid arthritis, Type
I diabetes and all autoimmune diseases combined compared with controls*

| | # of | Odds | Two-tailed |
|---|---|---|---|
| Disease group | subjects | Ratio (95% CI**) | p-value*** |
| Rheumatoid Arthritis | 234 | 8.31 (1.69 to 40.87) | 0.0056 |
| Type I diabetes | 178 | 9.34 (1.80 to 48.53) | 0.0064 |
| All autoimmune Disorders | 820 | 8.49 (1.98 to 36.34) | 0.0004 |

*Patients and controls (n = 648) were of European ancestry; Jewish subjects were not included in these analyses.
**95% CI = 95% Confidence Interval
***2 tailed p-value was determined using Fisher's exact test

TABLE 7
Specific disorders other than rheumatoid arthritis and Type I diabetes
analyzed for SIAE coding variants*

| | | Two-tailed |
|---|---|---|
| Disease group (n) | Odds Ratio (95% CI**) | p-value*** |
| Systemic lupus | 6.66 (1.22 to 39.49) | 0.046 |
| erythematosus (146) | | |
| Inflammatory Bowel | 7.48 (1.26 to 44.31) | 0.035 |
| Disease**** (130) | | |
| Multiple sclerosis (93) | 10.45 (1.77 to 61.73) | 0.016 |
| Juvenile Idiopathic arthritis | 9.81 (0.91 to 105.54) | 0.139 |
| (33) | | |
| Autoimmune thyroiditis | | |
| (2)***** | | |
| Sjogren's syndrome(1) | | |

*All patients and controls (n = 648) were of European ancestry; Jewish subjects were not included in these analyses.
**95% CI = 95% Confidence Interval
***Calculated using Fisher's exact test
****58 subjects with ulcerative colitis and 72 with Crohn's disease
*****27 patients with rheumatoid arthritis from the MADGC collection also had a second diagnosis of autoimmune thyroiditis

1. (canceled)

2. A method for treating an autoimmune disorder in a subject, the method comprising:
(a) identifying a subject in need of such treatment; and
(b) administering to the subject a therapeutically effective amount of an SIAE polypeptide.

3. The method of claim 2, wherein the SIAE polypeptide comprises the amino acid sequence of SEQ ID NO:1.

4. The method of claim 2, wherein the subject has a defect in an SIAE function.

5. The method of claim 2, wherein the subject has an SIAE gene that encodes a functionally defective SIAE polypeptide.

6. The method of claim 2, wherein the autoimmune disorder is selected from the group consisting of rheumatoid arthritis, type I diabetes, systemic lupus erythematosus, inflammatory bowel disease, multiple sclerosis, juvenile idiopathic arthritis, autoimmune thyroiditis, and Sjögren's syndrome.

7. The method of claim 2, further comprising determining whether the subject has a defect in an SIAE function by determining the presence of an SIAE variant allele that encodes a functionally defective SIAE protein, wherein the presence of the variant allele indicates that the subject has a defect in an SIAE function.

8. The method of claim 2, further comprising determining whether the subject has a defect in an SIAE function by obtaining a level of 9-O-acetyl sialic acid on B cells in the subject, wherein an increase in the level in the subject as compared to a level in a control subject indicates that the subject has a defect in an SIAE function.

9. The method of claim 2, further comprising determining whether the subject has a defect in an SIAE function by obtaining a serum level of SIAE protein in the subject, wherein a decrease in the level in the subject as compared to a level in a control subject indicates that the subject has a defect in an SIAE function.

10. A method for determining whether a subject is at risk for developing an autoimmune disorder, the method comprising:
(a) sequencing all or a part of a sialic acid acetylesterase (SIAE) gene of a subject;
(b) determining a subject amino acid sequence encoded by the SIAE gene in the subject;
(c) comparing the subject amino acid sequence to a reference SIAE amino acid sequence; and
(d) if there is an amino acid change in the subject amino acid sequence as compared to the reference SIAE amino acid sequence, determining an activity of an SIAE polypeptide comprising the subject amino acid sequence;
wherein a decrease in the activity indicates that the subject is at risk for developing an autoimmune disorder.

11. The method of claim 10, wherein the function of the SIAE polypeptide is esterase activity or secretion of the polypeptide.

12. The method of claim 10, wherein the subject is a human subject.

13. The method of claim 10, wherein the reference SIAE amino acid sequence is SEQ ID NO:1.

14. A method for determining whether a subject is at risk for developing an autoimmune disorder, the method comprising:
(a) providing a test sample from a subject; and
(b) assaying a level of 9-O-acetyl sialic acid on the surface of B cells in the test sample;
wherein an increase in the level in the test sample as compared to a level in a control sample indicates that the subject is at risk for developing an autoimmune disorder.

15. A method for determining whether a subject is at risk for developing an autoimmune disorder, the method comprising:
(a) providing a test serum or plasma sample from a subject; and
(b) assaying a level of SIAE protein in the sample;
wherein a decrease in the level in the test sample as compared to a level in a control sample indicates that the subject is at risk for developing an autoimmune disorder.
